SEQUENCE LISTING (26 sequences)

SEQ ID NO: 1 - protein, 37 aa, Felis catus
HDEFERHAEGTFTSDVSSYLEGQAAKEFIAWLVKGRG

SEQ ID NO: 2 - protein, 31 aa, Felis catus
HAEGTFTSDVSSYLEGQAAKEFIAWLVKGRG

SEQ ID NO: 3 - protein, 31 aa, Artificial Sequence
  Constructed sequence
HGEGTFTSDVSSYLEEQAAKEFIAWLVKGGG

SEQ ID NO: 4 - protein, 31 aa, Artificial Sequence
  Constructed sequence
HGEGTFTSDVSSYLEGQAAKEFIAWLVKGRG

SEQ ID NO: 5 - protein, 39 aa, Artificial Sequence
  Constructed sequence
HGEGTFTSDLSKQMEEEAVRLFIEWLKNGGPSSGAPPPS

SEQ ID NO: 6 - protein, 44 aa, Artificial Sequence
  Constructed sequence
HGEGTFTSDLSKQMEEEAVRLFIEWLKNGGPSSGAPPSKKKKKK

SEQ ID NO: 7 - protein, 43 aa, Felis catus
MAHIRGLWLPGCLALAALCSLVHSQHVFLAPQQALSLLQRVRR

SEQ ID NO: 8 - protein, 24 aa, Felis catus
MAHIRGLWLPGCLALAALCSLVHS

SEQ ID NO: 9 - protein, 19 aa, Felis catus
QHVFLAPQQALSLLQRVRR

SEQ ID NO: 10 - protein, 20 aa, Felis catus
MYKIQLLSCIALTLILVTNS

SEQ ID NO: 11 - protein, 237 aa, Felis catus
ARKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 12 - protein, 584 aa, Felis catus
EAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 13 - protein, 15 aa, Artificial Sequence
  Constructed sequence
GGGGSGGGGSGGGGS

SEQ ID NO: 14 - protein, 326 aa, Artificial Sequence
  Constructed sequence
MAHIRGLWLPGCLALAALCSLVHSQHVFLAPQQALSLLQRVRRHGEGTFTSDVSSYLEEQAAKEFIAWLVKGGGGGGGSGGGGSGGGGSARKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 15 - DNA, 981 bp, Artificial Sequence
  Constructed sequence
atggctcacatcagaggactttggctgcctggctgtctggctctggctgctctgtgttctctggtgcacagccagcacgtgtttctggcccctcagcaggctctgtccctgctgcaaagagttagaaggcacggcgagggcaccttcacctccgacgtgtctagctacctggaagaacaggccgccaaagagtttatcgcctggctggtcaaaggtggcggcggaggcggaggaagcggtggcggaggttcaggtggtggtggatctgccagaaagaccgaccatcctcctggacctaagccttgcgactgccctaagtgtcctccacctgagatgctcggcggacccagcatcttcatcttcccacctaagccaaaggacaccctgagcatcagcagaacccctgaagtgacctgcctggtcgttgatctgggccccgacgatagcgacgtgcagatcacttggtttgtggacaacacccaggtgtacacagccaagacaagccccagagaggaacagttcaacagcacctacagagtggtgtccgtgctgcccatcctgcaccaggattggctgaagggcaaagaattcaagtgcaaagtgaacagcaagagcctgccttctccaatcgagcggaccatcagcaaggccaagggacagcctcacgagcctcaggtgtacgtcctgcctcctgctcaagaggaactgagccggaacaaagtgtccgtgacctgtctgatcaagagctttcacccacctgatatcgccgtggaatgggagatcacaggccagcctgagcctgagaacaactaccggactacccctccacagctggactccgatggcacctacttcgtgtacagcaagctgagcgtggacagaagccactggcagcggggcaatacctacacctgttccgtgtctcacgaggccctgcacagccaccacacacagaagtctctgacacagagccccggctga

SEQ ID NO: 16 - protein, 670 aa, Artificial Sequence
  Constructed sequence
MAHIRGLWLPGCLALAALCSLVHSQHVFLAPQQALSLLQRVRRHGEGTFTSDVSSYLEEQAAKEFIAWLVKGGGGSGGGGSGGGASEAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 17 - DNA, 2016 bp, Artificial Sequence
  Constructed sequence
atggctcacatcagaggactttggctgcctggctgtctggctctggctgctctgtgttctctggtgcacagccagcacgtgtttctggcccctcagcaggctctgtccctgctgcaaagagttagaaggcacggcgagggcaccttcacctccgacgtgtccagctacctggaagaacaggccgccaaagagtttatcgcctggctggtcaaaggcggcggaggatctggcggaggtggaagcggaggcggagcttctgaagcccaccagtctgagatcgcccaccggttcaacgacctgggcgaagaacacttcagaggcctggtgctggtggcctttagccagtacctgcagcagtgccccttcgaggatcacgtgaagctggtcaacgaagtgaccgagttcgccaagggctgtgtggccgatcagtctgccgccaattgcgagaagtctctgcacgagctgctgggcgataagctgtgtacagtggccagcctgcgggataagtacggcgagatggccgactgctgcgagaagaaagagcccgagagaaacgagtgcttcctgcagcacaaggacgacaaccctggctttggccagctggttacacctgaggccgacgccatgtgcaccgccttccacgagaacgagcagagattcctgggcaagtacctgtacgagatcgccagacggcacccctacttttacgcccctgagctgctgtactacgccgaagagtacaagggcgtgttcaccgagtgttgcgaggccgctgataaggccgcttgtctgacacccaaagtggacgccctgagagaaaaggtgctggcctccagcgccaaagaaagactgaagtgcgcctctctgcaaaagttcggcgagagagccttcaaggcttggagcgtggcaagactgagccagaagttccccaaggccgagtttgccgagatcagcaagctcgtgaccgacctggccaagatccacaaagagtgctgccacggcgacctgctggaatgcgctgacgatagagccgatctggctaagtacatctgcgagaaccaggacagcatcagcaccaagctgaaagagtgctgcggcaagcccgtgctggaaaagagccactgtatcagcgaggtggaacgggacgagctgcctgctgatctgcctcctctggccgtggacttcgtcgaggacaaagaagtgtgcaagaactaccaagaggccaaggacgtgttcctgggaacctttctgtacgagtactctcggcggcaccccgagtattccgttagcctgctgctgcggctggcaaaagagtacgaggccacactggaaaagtgctgcgccaccgacgatcctccagcctgttacgcccacgtgttcgacgagttcaagcccctggtggaagaaccccacaacctggtcaagaccaactgcgaactgttcgagaagctgggcgagtacggcttccagaacgccctgctcgtgcggtacaccaagaaggtgccccaggtttccacacctacactggtcgaggtgtcccgctctctgggcaaagtgggcagcaagtgctgtacacacccagaggccgagagactgagctgcgccgaggattatctgagcgtggtgctgaacagactgtgcgtgctgcacgagaaaacccctgtgtctgagcgcgtgaccaagtgttgtaccgagagcctggtcaacagacggccttgctttagcgccctgcaagtcgacgagacatacgtgccaaaagagttcagcgccgagacattcaccttccacgccgacctgtgtacactgcccgaggccgaaaagcagatcaagaaacagagcgccctggtcgaactgctgaagcacaagcctaaggccaccgaggaacagctgaaaaccgtgatgggcgacttcggcagcttcgtggataagtgctgtgccgctgaggacaaagaggcctgcttcgctgaagagggccctaagttggttgccgctgctcaagctgccctggcctgataa

SEQ ID NO: 18 - protein, 687 aa, Artificial Sequence
  Constructed sequence
MAHIRGLWLPGCLALAALCSLVHSQHVFLAPQQALSLLQRVRRHGEGTFTSDVSSYLEGQAAKEFIAWLVKGRHGEGTFTSDVSSYLEGQAAKEFIAWLVKGREAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 19 - DNA, 2067 bp, Artificial Sequence
  Constructed sequence
atggctcacatcagaggactttggctgcctggctgtctggctctggctgctctgtgttctctggtgcacagccagcacgtgtttctggcccctcagcaggctctgtccctgctgcaaagagttagaaggcacggcgagggcaccttcacctccgacgtgtctagctacctggaaggacaggccgccaaagagtttatcgcctggctggtcaaaggcagacacggcgaagggacattcacaagcgacgtgtcctcttatctcgaaggccaggctgctaaagagttcattgcttggctcgtgaagggcagagaggcccaccagtctgagatcgcccacagattcaacgacctgggcgaagaacacttcagaggcctggtgctggtggccttcagccagtatctgcagcagtgccccttcgaggaccacgtgaagctggtcaacgaagtgaccgagttcgccaagggctgtgtggccgatcagtctgccgccaattgcgagaagtctctgcacgagctgctgggcgataagctgtgtacagtggccagcctgcgggataagtacggcgagatggccgactgctgcgagaagaaagagcccgagagaaacgagtgcttcctgcagcacaaggacgacaaccctggctttggccagctggttacacctgaggccgacgccatgtgcaccgccttccacgagaacgagcagagattcctgggcaagtacctgtacgagatcgccagacggcacccctacttttacgcccctgagctgctgtactacgccgaagagtacaagggcgtgttcaccgagtgttgcgaggccgctgataaggccgcttgtctgacacccaaagtggacgccctgagagaaaaggtgctggcctccagcgccaaagaaagactgaagtgcgcctctctgcaaaagttcggcgagagagccttcaaggcttggagcgtggcaagactgagccagaagttccccaaggccgagtttgccgagatcagcaagctcgtgaccgacctggccaagatccacaaagagtgctgccacggcgacctgctggaatgcgctgacgatagagccgatctggctaagtacatctgcgagaaccaggacagcatcagcaccaagctgaaagagtgctgcggcaagcccgtgctggaaaagagccactgtatcagcgaggtggaacgggacgagctgcctgctgatctgcctcctctggccgtggacttcgtcgaggacaaagaagtgtgcaagaactaccaagaggccaaggacgtgttcctgggaacctttctgtacgagtactctcggcggcaccccgagtattccgttagcctgctgctgcggctggcaaaagagtacgaggccacactggaaaagtgctgcgccaccgacgatcctccagcctgttacgcccacgtgttcgacgagttcaagcccctggtggaagaaccccacaacctggtcaagaccaactgcgaactgttcgagaagctgggcgagtacggcttccagaacgccctgctcgtgcggtacaccaagaaggtgccccaggtttccacacctacactggtcgaggtgtcccgctctctgggcaaagtgggcagcaagtgctgtacacacccagaggccgagagactgagctgcgccgaggattatctgagcgtggtgctgaacagactgtgcgtgctgcacgagaaaacccctgtgtctgagcgcgtgaccaagtgttgtaccgagagcctggtcaacagacggccttgctttagcgccctgcaagtcgacgagacatacgtgccaaaagagttcagcgccgagacattcaccttccacgccgacctgtgtacactgcccgaggccgaaaagcagatcaagaaacagagcgccctggtcgaactgctgaagcacaagcctaaggccaccgaggaacagctgaaaaccgtgatgggcgacttcggcagcttcgtggataagtgctgtgccgctgaggacaaagaggcctgcttcgctgaagagggccctaagttggttgccgctgctcaagctgccctggcctgataa

SEQ ID NO: 20 - protein, 657 aa, Artificial Sequence
  Constructed sequence
MAHIRGLWLPGCLALAALCSLVHSQHVFLAPQQALSLLQRVRRHGEGTFTSDVSSYLEGQAAKEFIAWLVKGREAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 21 - DNA, 3342 bp, Artificial Sequence
  Constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacatcagaggactttggctgcctggctgtctggctctggctgctctgtgttctctggtgcacagccagcacgtgtttctggcccctcagcaggctctgtccctgctgcaaagagttagaaggcacggcgagggcaccttcacctccgacgtgtctagctacctggaagaacaggccgccaaagagtttatcgcctggctggtcaaaggtggcggcggaggcggaggaagcggtggcggaggttcaggtggtggtggatctgccagaaagaccgaccatcctcctggacctaagccttgcgactgccctaagtgtcctccacctgagatgctcggcggacccagcatcttcatcttcccacctaagccaaaggacaccctgagcatcagcagaacccctgaagtgacctgcctggtcgttgatctgggccccgacgatagcgacgtgcagatcacttggtttgtggacaacacccaggtgtacacagccaagacaagccccagagaggaacagttcaacagcacctacagagtggtgtccgtgctgcccatcctgcaccaggattggctgaagggcaaagaattcaagtgcaaagtgaacagcaagagcctgccttctccaatcgagcggaccatcagcaaggccaagggacagcctcacgagcctcaggtgtacgtcctgcctcctgctcaagaggaactgagccggaacaaagtgtccgtgacctgtctgatcaagagctttcacccacctgatatcgccgtggaatgggagatcacaggccagcctgagcctgagaacaactaccggactacccctccacagctggactccgatggcacctacttcgtgtacagcaagctgagcgtggacagaagccactggcagcggggcaatacctacacctgttccgtgtctcacgaggccctgcacagccaccacacacagaagtctctgacacagagccccggctgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 4411 bp, Artificial Sequence
  Constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggctcacatcagaggactttggctgcctggctgtctggctctggctgctctgtgttctctggtgcacagccagcacgtgtttctggcccctcagcaggctctgtccctgctgcaaagagttagaaggcacggcgagggcaccttcacctccgacgtgtctagctacctggaaggacaggccgccaaagagtttatcgcctggctggtcaaaggcagacacggcgaagggacattcacaagcgacgtgtcctcttatctcgaaggccaggctgctaaagagttcattgcttggctcgtgaagggcagagaggcccaccagtctgagatcgcccacagattcaacgacctgggcgaagaacacttcagaggcctggtgctggtggccttcagccagtatctgcagcagtgccccttcgaggaccacgtgaagctggtcaacgaagtgaccgagttcgccaagggctgtgtggccgatcagtctgccgccaattgcgagaagtctctgcacgagctgctgggcgataagctgtgtacagtggccagcctgcgggataagtacggcgagatggccgactgctgcgagaagaaagagcccgagagaaacgagtgcttcctgcagcacaaggacgacaaccctggctttggccagctggttacacctgaggccgacgccatgtgcaccgccttccacgagaacgagcagagattcctgggcaagtacctgtacgagatcgccagacggcacccctacttttacgcccctgagctgctgtactacgccgaagagtacaagggcgtgttcaccgagtgttgcgaggccgctgataaggccgcttgtctgacacccaaagtggacgccctgagagaaaaggtgctggcctccagcgccaaagaaagactgaagtgcgcctctctgcaaaagttcggcgagagagccttcaaggcttggagcgtggcaagactgagccagaagttccccaaggccgagtttgccgagatcagcaagctcgtgaccgacctggccaagatccacaaagagtgctgccacggcgacctgctggaatgcgctgacgatagagccgatctggctaagtacatctgcgagaaccaggacagcatcagcaccaagctgaaagagtgctgcggcaagcccgtgctggaaaagagccactgtatcagcgaggtggaacgggacgagctgcctgctgatctgcctcctctggccgtggacttcgtcgaggacaaagaagtgtgcaagaactaccaagaggccaaggacgtgttcctgggaacctttctgtacgagtactctcggcggcaccccgagtattccgttagcctgctgctgcggctggcaaaagagtacgaggccacactggaaaagtgctgcgccaccgacgatcctccagcctgttacgcccacgtgttcgacgagttcaagcccctggtggaagaaccccacaacctggtcaagaccaactgcgaactgttcgagaagctgggcgagtacggcttccagaacgccctgctcgtgcggtacaccaagaaggtgccccaggtttccacacctacactggtcgaggtgtcccgctctctgggcaaagtgggcagcaagtgctgtacacacccagaggccgagagactgagctgcgccgaggattatctgagcgtggtgctgaacagactgtgcgtgctgcacgagaaaacccctgtgtctgagcgcgtgaccaagtgttgtaccgagagcctggtcaacagacggccttgctttagcgccctgcaagtcgacgagacatacgtgccaaaagagttcagcgccgagacattcaccttccacgccgacctgtgtacactgcccgaggccgaaaagcagatcaagaaacagagcgccctggtcgaactgctgaagcacaagcctaaggccaccgaggaacagctgaaaaccgtgatgggcgacttcggcagcttcgtggataagtgctgtgccgctgaggacaaagaggcctgcttcgctgaagagggccctaagttggttgccgctgctcaagctgccctggcctgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 4360 bp, Artificial Sequence
  Constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggctcacatcagaggactttggctgcctggctgtctggctctggctgctctgtgttctctggtgcacagccagcacgtgtttctggcccctcagcaggctctgtccctgctgcaaagagttagaaggcacggcgagggcaccttcacctccgacgtgtccagctacctggaagaacaggccgccaaagagtttatcgcctggctggtcaaaggcggcggaggatctggcggaggtggaagcggaggcggagcttctgaagcccaccagtctgagatcgcccaccggttcaacgacctgggcgaagaacacttcagaggcctggtgctggtggcctttagccagtacctgcagcagtgccccttcgaggatcacgtgaagctggtcaacgaagtgaccgagttcgccaagggctgtgtggccgatcagtctgccgccaattgcgagaagtctctgcacgagctgctgggcgataagctgtgtacagtggccagcctgcgggataagtacggcgagatggccgactgctgcgagaagaaagagcccgagagaaacgagtgcttcctgcagcacaaggacgacaaccctggctttggccagctggttacacctgaggccgacgccatgtgcaccgccttccacgagaacgagcagagattcctgggcaagtacctgtacgagatcgccagacggcacccctacttttacgcccctgagctgctgtactacgccgaagagtacaagggcgtgttcaccgagtgttgcgaggccgctgataaggccgcttgtctgacacccaaagtggacgccctgagagaaaaggtgctggcctccagcgccaaagaaagactgaagtgcgcctctctgcaaaagttcggcgagagagccttcaaggcttggagcgtggcaagactgagccagaagttccccaaggccgagtttgccgagatcagcaagctcgtgaccgacctggccaagatccacaaagagtgctgccacggcgacctgctggaatgcgctgacgatagagccgatctggctaagtacatctgcgagaaccaggacagcatcagcaccaagctgaaagagtgctgcggcaagcccgtgctggaaaagagccactgtatcagcgaggtggaacgggacgagctgcctgctgatctgcctcctctggccgtggacttcgtcgaggacaaagaagtgtgcaagaactaccaagaggccaaggacgtgttcctgggaacctttctgtacgagtactctcggcggcaccccgagtattccgttagcctgctgctgcggctggcaaaagagtacgaggccacactggaaaagtgctgcgccaccgacgatcctccagcctgttacgcccacgtgttcgacgagttcaagcccctggtggaagaaccccacaacctggtcaagaccaactgcgaactgttcgagaagctgggcgagtacggcttccagaacgccctgctcgtgcggtacaccaagaaggtgccccaggtttccacacctacactggtcgaggtgtcccgctctctgggcaaagtgggcagcaagtgctgtacacacccagaggccgagagactgagctgcgccgaggattatctgagcgtggtgctgaacagactgtgcgtgctgcacgagaaaacccctgtgtctgagcgcgtgaccaagtgttgtaccgagagcctggtcaacagacggccttgctttagcgccctgcaagtcgacgagacatacgtgccaaaagagttcagcgccgagacattcaccttccacgccgacctgtgtacactgcccgaggccgaaaagcagatcaagaaacagagcgccctggtcgaactgctgaagcacaagcctaaggccaccgaggaacagctgaaaaccgtgatgggcgacttcggcagcttcgtggataagtgctgtgccgctgaggacaaagaggcctgcttcgctgaagagggccctaagttggttgccgctgctcaagctgccctggcctgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 2211 bp, adeno-associated virus rh91
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 25 - DNA, 2211 bp, adeno-associated virus rh91
  misc_feature (1)..(2211): AAVrh.91
  misc_feature (1)..(2211): AAVrh91
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 26 - protein, 736 aa, adeno-associated virus rh91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

(19) United States
(12) Patent Application Publication
(10) Pub. No.: US 2023/0405150 A1
(43) Pub. Date: Dec. 21, 2023

(54) VIRAL VECTOR ENCODING GLP-1 RECEPTOR AGONIST FUSIONS AND USES THEREOF IN TREATING METABOLIC DISEASES IN FELINES

(71) Applicant: The Trustees of the University of Pennsylvania, Philadelphia, PA (US)

(72) Inventors: James M. Wilson, Philadelphia, PA (US); Christian Hinderer, Baltimore, MD (US); Makoto Horiuchi, Wallingford, PA (US)

(73) Assignee: The Trustees of the University of Pennsylvania, Philadelphia, PA (US)

(21) Appl. No.: 18/042,729

(22) PCT Filed: Aug. 24, 2021
(86) PCT No.: PCT/US2021/047403

§ 371 Date: Feb. 23, 2023

Related U.S. Application Data
(60) Provisional application No. 63/069,492, filed on Aug. 24, 2020.

(51) Int. Cl. A61K 48/00; C12N 15/86; C07K 14/605; C12N 9/48; A61P 3/10; C07K 14/76; C07K 14/55

(52) U.S. Cl. CPC A61K 48/005; C12N 15/86; C07K 14/605; C12N 9/485; A61K 38/00; C07K 14/76; C07K 14/55; C12Y 304/14005; A61P 3/10

(57) ABSTRACT

Compositions and methods for treating type II diabetes in a feline are provided. A viral vector is provided which includes a nucleic acid molecule comprising a sequence encoding a feline GLP-1 receptor agonist.

### BACKGROUND OF THE INVENTION

One in 400 cats and 1 in 500 dogs in the U.S. have a condition similar to diabetes in humans. The current standard of care is twice daily insulin injections by the owner along with frequent veterinarian visits and disposable diagnostics that are expensive, time consuming and inconvenient for the owners of these animals.

Type II diabetes (T2DM) is the most common form seen in cats, accounting for ˜90% of cases. Risk factors include increased age, male gender, obesity, indoor confinement, physical inactivity, breed, and long-acting or repeated steroid or megestrol acetate administration. These factors lead to decreased insulin sensitivity, and increase the demand on β-cells to produce insulin. Gottleib and Rand, Managing feline diabetes: current perspectives, Veterinary Medicine: Research and Reports, June 2018:9 33-42.

Glucagon-like peptide 1 (GLP-1) is an endogenous peptide hormone that plays a central role in glucose homeostasis. GLP-1 receptor agonists are currently used in humans for the treatment of diabetes. GLP-1 and other GLP-1 receptor agonists have the ability to control hyperglycemia by potentiating insulin release, increasing insulin sensitivity, preventing beta cell loss, and delaying gastric emptying. GLP-1 receptor agonists engineered to overcome the short half-life of the native hormone by fusing the agonist to a protein with longer half-life have emerged as important therapeutics for the treatment of T2DM.

### SUMMARY OF THE INVENTION

Viral vectors encoding glucagon-like peptide 1 (GLP-1) receptor agonist fusion proteins adapted for use in felines are provided herein. These viral vectors may achieve, in some embodiments, sustained expression of the GLP-1 receptor agonist in felines and/or increased half-life compared to vector-mediated delivery of a GLP-1 receptor agonist without a fusion partner or compared with a fusion partner not adapted for use in felines. Further provided are methods of making and using such viral vectors.

In one aspect, a viral vector is provided which includes a nucleic acid comprising a polynucleotide sequence encoding a fusion protein. The fusion protein includes (a) a leader sequence comprising a secretion signal peptide, (b) a glucagon-like peptide-1 (GLP-1) receptor agonist, and (c) a fusion domain comprising either (i) a feline IgG Fc or a functional variant thereof or (ii) a feline albumin or a functional variant thereof. In one embodiment, the vector is an adeno-associated viral vector.

In on embodiment, the (i) the secretion signal peptide of the leader sequence comprises a feline thrombin signal peptide; (ii) the leader sequence comprises a feline thrombin propeptide; and/or (iii) the leader sequence comprises a feline thrombin leader sequence. In another embodiment, the leader sequence comprises a feline IL-2 leader sequence. In one embodiment, the GLP-1 receptor agonist is selected from SEQ ID NO: 1, SEQ ID NO: 2, SEQ ID NO: 3, SEQ ID NO: 4, SEQ ID NO: 5, SEQ ID NO: 6, and functional variants thereof.

In one embodiment, the fusion domain is a feline IgG Fc having the sequence of SEQ ID NO: 11, or a sequence sharing at least 90% identity therewith, or a functional variant thereof. In another embodiment, the fusion domain is a feline albumin having the sequence of SEQ ID NO: 12, or a sequence sharing at least 90% identity therewith, or a functional variant thereof.

In another aspect, the viral vector includes an AAV capsid, and a vector genome packaged in the AAV capsid, said vector genome comprising AAV inverted terminal repeats (ITRs), the polynucleotide sequence encoding the fusion protein, and regulatory sequences which direct expression of the fusion protein.

In another aspect, a pharmaceutical composition suitable for use in treating a metabolic disease in a feline is provided. The composition includes an aqueous liquid and the viral vector as described herein.

In yet another aspect, use of a viral vector as described herein is provided for the manufacture of a medicament for treating a feline subject having a metabolic disease, optionally diabetes.

In another aspect, a method of treating a feline subject having a metabolic disease is provided. The method includes administering to the feline subject an effective amount of a viral vector or composition as described herein,

Other aspects and advantages of the invention will be readily apparent from the following detailed description of the invention.

### BRIEF DESCRIPTION OF THE DRAWINGS

FIG. 1A is a schematic drawing of Dulaglutide.

FIG. 1B is a schematic drawing of Albiglutide.

FIG. 2A and FIG. 2B are bar graphs showing GLP-1 expression in HEK293 cells. HEK293 cells were transfected with plasmids expressing feline GLP1R agonists and active GLP-1 expression was measured by ELISA specific to active form GLP-1 (7-36) at 48 h post transfection (FIG. 2A). FIG. 1B shows the response ratio as measured against control. GLP-1 activity in the culture supernatants was measured by cell-based GLP-1 activity assay (GeneBLAzer GLP1R-CRE-bla CHO-KI cell-based assay). Fusion protein construct with feTrb.feGLP1-IgG Fc (CB7.feDulaglutide(feTrb).rBG) showed highest expression/activity of constructs tested.

FIG. 3A and FIG. 3B show pilot expression of GLP-1 in Rag1KO mice. Rag1KO female mice were dosed with 1×10¹¹ GC/mouse via I.M. of the shown vectors. Weekly bleeds were performed. GLP-1 ELISA and GLP-1 activity assays were performed. FIG. 3A shows the results of a GLP-1 ELISA performed from weekly bleeds of the treated mice. FIG. 3B shows serum GLP-1 activity at day 21 after injection.

FIG. 4A-FIG. 4E show the results of a feline study performed with AAVrh91.CB7.CI.feDulaglutide (feTrbss) and pAAV.CB7.CI.feGLP1-SA (feTrb).RBG. Cats were treated with various doses of vectors via I.M. injection, and transgene expression and body weight were recorded for at least 28 days after injection. FIG. 4A shows weekly body weights of individual cats treated with 5×10¹¹ GC/kg of AAVrh91.CB7.CI.feDulaglutide (feTrbss) through to week 17. FIG. 4B shows the corresponding serum levels do fe-GLP-1-Fc for cats from FIG. 4A (shown as mean+/−SD).

FIG. 4C shows the relationship between AAV dose and serum feGLP-1-Fc levels 28 days after IM administration of AAVrh91.CB7.CI.feDulaglutide (feTrbss) at either, 5×10¹¹ GC/kg, 1×10¹¹ GC/cat, 1×10¹⁰ GC/cat or 1×10⁹ GC/cat. (Data shown is mean+/−SD, n+4/group). FIG. 4D shows the relative expression over 28 days of fe-GLP-1-SA in cats administered 1×10¹¹ GC/cat of pAAV.CB7.CI.feGLP1-SA (feTrb).RBG (mean+/−SD, n=4). FIG. 4E is a comparison of the activity of fe-GLP-1 proteins in the serum of cats at Day 28 from the cohorts described above. FIG. 4F shows body weight loss [ ]. FIG. 4G shows feGLP1Fc expression in animals.

FIG. 5 is a plasmid map of pAAV.CB7.CI.feDulaglutide (feTrbss).RBG.

FIG. 6 is a plasmid map of pAAV.CB7.CI.feAlbiglutide (feTrb).RBG.

FIG. 7 is a plasmid map of pAAV.CB7.CI.feGLP1-SA (feTrb).RBG.

FIG. 8A shows an amino acid sequence comprising feline Dulaglutide with feline thrombin signal sequence (SEQ ID NO: 14).

FIG. 8B shows an amino acid sequence comprising feline Albiglutide with feline thrombin signal sequence (SEQ ID NO: 18).

FIG. 8C shows an amino acid sequence comprising feline GLP1-SA with feline thrombin signal sequence (SEQ ID NO: 16).

FIG. 9 shows a graph of serum concentration of fGLP-1-SA at study days 0-182 after intramuscular injection of AAV feGLP-1-SA. Dotted line indicates target therapeutic threshold.

FIG. 10 shows a graph of serum concentrations of feGLP-1-Fc for 330 days after intramuscular injection of AAV feGLP-1-Fc.

FIG. 11 shows a graph of anti-transgene product antibody response in an AAV fGLP-1-Fc treated cat.

FIG. 12 shows a graph of expression of fGLP-1-SA for 336 days after intramuscular injection of AAV feGLP-1-SA (n=4 per group).

### DETAILED DESCRIPTION OF THE INVENTION

Long-acting GLP-1 receptor agonist fusion protein expression constructs have been developed for use in feline animals. A leader sequence is provided which includes a secretion signal peptide, as well as a fusion domain intended to prolong the time in circulation of the resulting fusion protein.

The compositions and methods described herein are intended to be for use in feline animals. The term feline (family Felidae) refers to any of 37 cat species that among others include the cheetah, puma, jaguar, leopard, lion, lynx, tiger, and domestic cat. In a preferred embodiment, the subject is a domestic cat.

Delivery of these constructs to subjects in need thereof via a number of routes, and particularly by expression in vivo mediated by a recombinant vector, such as a rAAV vector, is described. Also provided are methods of using these constructs in regimens for treating T2DM or metabolic syndrome in a veterinary subject in need thereof and increasing the half-life of GLP-1 in a subject. In addition, methods are provided for enhancing the activity of GLP-1 in a subject. Also provided are methods for inducing weight loss in a veterinary subject in need thereof.

Glucagon-like peptide 1, or GLP-1, is an incretin derived from the transcription product of the proglucagon gene. In vivo, the glucagon gene expresses a 180 amino acid prepropolypeptide that is proteolytically processed to form glucagon, two forms of GLP-1 and GLP-2. The original sequencing studies indicated that GLP-1 possessed 37 amino acid residues. However, subsequent information showed that this peptide was a propeptide and was additionally processed to remove 6 amino acids from the amino-terminus to a form GLP-1(7-37), an active form of GLP-1. The glycine at position 37 is also transformed to an amide in vivo to form GLP-1 (7-36) amide. GLP-1 (7-37) and GLP-1 (7-36) amide are insulinotropic hormones of equal potency. Thus, as used herein, the biologically “active” forms of GLP-1 which are useful herein are: GLP-1-(7-37) and GLP-1-(7-36)NH₂.

GLP-1 receptor agonists are a class of antidiabetic agents that mimic the action of the glucagon-like peptide. GLP-1 is one of several naturally occurring incretin compounds that affect the body after they are released from the gut during digestion. By binding and activating GLP-1 receptors, GLP-1 receptor agonists are able to reduce blood glucose levels helping T2DM patients to reach a glycemic control. As used herein the term “GLP-1 receptor agonist” refers to GLP-1 or a functional fragment thereof, amino-acid sequence variants of GLP-1 or functional fragments thereof, and other polypeptide agonists for the GLP-1 receptor (e.g. exedin-4 and variants thereof). The disclosure provides fusion proteins comprising one or more copies of a GLP-1 receptor agonist, as well as polynucleotides and vectors encoding such fusion proteins. In some embodiments, the fusion protein comprises a polynucleotide sequence encoding a fusion protein comprising (a) a leader sequence comprising a secretion signal peptide, (b) a glucagon-like peptide-1 (GLP-1) receptor agonist, and (c) a fusion domain comprising either (i) a feline IgG Fc or a functional variant thereof or (ii) a feline albumin or a functional variant thereof. In one embodiment, the fusion protein comprises a feline thrombin leader sequence, a GLP-1 receptor agonist, and a feline IgG Fc or functional variant thereof. In another embodiment, the fusion protein comprises a feline thrombin leader, a GLP-1 receptor agonist, and a feline albumin or functional variant thereof. In another embodiment, the fusion protein comprises a feline thrombin leader, two copies of a GLP-1 receptor agonist, and a feline albumin or functional variant thereof. In another embodiment, the fusion protein comprises a feline thrombin leader, two copies of a GLP-1 receptor agonist, and a feline IgG Fc or functional variant thereof.

In some embodiments, GLP-1 receptor agonists include variants which may include up to about 10% variation from a GLP-1 nucleic acid or amino acid sequence described herein or known in the art, which retain the function of the wild-type sequence. As used herein, by “retain function” it is meant that the nucleic acid or amino acid functions in the same way as the wild type sequence, although not necessarily at the same level of expression or activity. For example, in one embodiment, a functional variant has increased expression or activity as compared to the wild type sequence. In another embodiment, the functional variant has decreased expression or activity as compared to the wild type sequence. In one embodiment, the functional variant has 10%, 20%, 30%, 40%, 50%, 60%, 70%, 80%, 90% or greater increase or decrease in expression or activity as compared to the wild type sequence.

Several human drugs that fuse a GLP-1 receptor agonist to a stabilizing fusion domain are known in the art. These include albiglutide, liraglutide, dulaglutide and lixisenatide (also known by its chemical name des-38-proline-exendin-4 (Heloderma suspectum)-(1-39)-peptidylpenta-L-lysyl-L-lysinamide). In the present disclosure, the term for the human drug preceded by the prefix “fe” refers to a variant of the human drug in which the human fusion domain is replaced with the feline homolog of that fusion domain and, where the GLP-1 receptor agonist is a fragment or variant of a human protein, the GLP-1 receptor agonist is replaced with the feline homolog of that fragment or variant.

Dulaglutide is a disulfide-bonded homodimer fusion peptide with each monomer consisting of one GLP-1 analog moiety and one IgG4 Fc region. Yu M, et al. (2018) Battle of GLP-1 delivery technologies, Adv. Drug Deliv. Rev. A schematic of dulaglutide is shown in FIG. TA. See, WO 2005/000892A2, which is incorporated herein by reference.

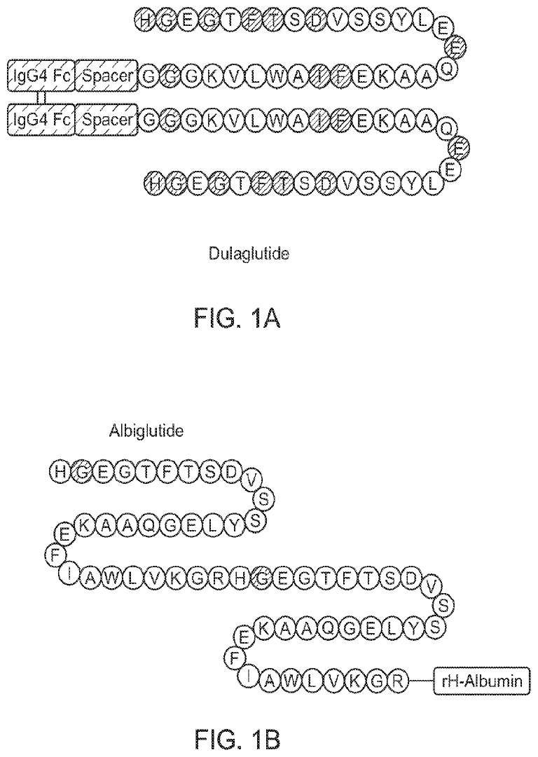

Albiglutide is a recombinant protein composed of two copies of GLP-1 analogs fused to human albumin. The molecule has a Gly8 to Ala substitution in both copies of the GLP-1 analogs to improve resistance to DPP-4 degradation. A schematic of albiglutide is shown in FIG. 1B.

The fusion comprises, in one embodiment, a GLP-1 analog in combination with feline heterologous sequences. By GLP-1 analog is meant a polypeptide sharing at least 90%, 95%, 97%, 98%, 99% or 100% identity with native feline GLP-1(7-37). In one embodiment, the GLP-1 analog has at most 1, 2, or 3 amino acid substitutions as compared to the native sequence. Native feline GLP-1(1-37) has the sequence of HDEFERHAEGTFTSDVSSYLEGQAAKEFIAWLVKGRG (SEQ ID NO: 1), with GLP-1(7-37) having the sequence of HAEGTFTSDVSSYLEGQAAKEFIAWLVKGRG (SEQ ID NO: 2). In some embodiments, it is desirable to alter the native GLP-1 sequence to optimize one or more features thereof. For example, in one embodiment, the GLP-1 analog contains one, two, or three amino acid substitutions selected from A8G, G22E, and R36G as compared to the native sequence (using the full-length native GLP-1 numbering as reference). For clarity, with respect to GLP-1(7-37), these amino acid substitutions are A2G, G16E, and R30G. These substitutes have been shown to improve efficacy of the clinical profile of GLP-1, including protection from DPP-4 inactivation (A8G), increased solubility (G22E), and reduction of immunogenicity via substituting a glycine residue for arginine at position 36 (R36G) to remove a potential T-cell epitope. In one embodiment, the GLP-1 analog is a DPP-IV resistant variant of feline GLP-1. In one embodiment, the GLP-1 analog has a sequence comprising, or consisting of, SEQ ID NO: 3: HGEGTFTSDVSSYLEEQAAKEFIAWLVKGGG. In another embodiment, the GLP-1 analog has a sequence comprising, or consisting of, SEQ ID NO: 4: HGEGTFTSDVSSYLEGQAAKEFIAWLVKGRG. In another embodiment, the GLP-1 receptor agonist has a sequence comprising, or consisting, of SEQ ID NO: 5: HGEGTFTSDLSKQMEEEAVRLFIEWLKNGGPSSGAPPPS (exendin-4) or a functional variant thereof. In one embodiment, the variant shares at least 90% identity, 95% identity, 97% identity, 98% identity, 99% identity or 100% identity with SEQ ID NO: 5. In another embodiment, the GLP-1 receptor agonist has a sequence comprising, or consisting, of SEQ ID NO: 6: HGEGTFTSDLSKQMEEEAVRLFIEWLKNGGPSSGAPPSKKKKKK or a functional variant thereof. In one embodiment, the variant shares at least 90% identity, 95% identity, 97% identity, 98% identity, 99% identity or 100% identity with SEQ ID NO: 6. In one embodiment, more than one copy of the GLP-1 analog is present in the fusion protein. In another embodiment, the GLP-1 receptor agonist is two tandem copies of GLP-1(7-37) or a DPP-IV resistant variant thereof.

The fusion protein may comprise a leader sequence, which may comprise a secretion signal peptide. As used herein, the term “leader sequence” refers to any N-terminal sequence of a polypeptide.

The leader sequence may be derived from the same species for which administration is ultimately intended, i.e., a feline animal. As used herein, the terms “derived” or “derived from” mean the sequence or protein is sourced from a specific subject species or shares the same sequence as a protein or sequence sourced from a specific subject species. For example, a leader sequence which is “derived from” a feline, shares the same sequence (or a variant thereof, as defined herein) as the same leader sequence as expressed in a feline. However, the specified nucleic acid or amino acid need not actually be sourced from a feline. Various techniques are known in the art which are able to produce a desired sequence, including mutagenesis of a similar protein (e.g., a homolog) or artificial production of a nucleic acid or amino acid sequence. The “derived” nucleic acid or amino acid retains the function of the same nucleic acid or amino acid in the species from which it is “derived”, regardless of actual source of the derived sequence.

The term “amino acid substitution” and its synonyms are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

In one embodiment, the leader is a feline thrombin (Factor II) sequence. In one embodiment, the thrombin leader has the sequence shown in SEQ ID NO: 7: MAHIRGLWLPGCLALAALCSLVHSQHVFLAPQQALSLLQRVRR, or a functional variant thereof having at most 1, 2, or 3 amino acid substitutions. In some embodiments, the leader comprises a signal peptide and a propeptide. In one embodiment, the secretion signal peptide of the leader sequence comprises a feline thrombin signal peptide. In one embodiment, the signal peptide is MAHIRGLWLPGCLALAALCSLVHS (SEQ ID NO: 8) or a functional variant thereof having at most 1, 2, or 3 amino acid substitutions. In another embodiment, the leader sequence comprises a feline thrombin propeptide. In one embodiment, the propeptide has the sequence of QHVFLAPQQALSLLQRVRR (SEQ ID NO: 9) or a functional variant thereof having at most 1, 2, or 3 amino acid substitutions.

In one embodiment, the leader is a feline IL-2 sequence. In one embodiment, the IL-2 leader has the sequence shown in SEQ ID NO: 10: MYKIQLLSCIALTLILVTNS, or a functional variant thereof having at most 1, 2, or 3 amino acid substitutions.

In one embodiment, functional variants of the desired leader include variants which may include up to about 10% variation from a leader nucleic acid or amino acid sequence described herein or known in the art, which retain the function of the wild type sequence.

In some embodiments, the coding regions for both the propeptide and GLP-1 peptide are incorporated into a single nucleic acid sequence without a linker between the coding sequences of the propeptide and GLP-1.

The fusion protein further includes a fusion domain. The fusion domain, in one embodiment, is a feline IgG Fc fragment (e.g., IgG1a, IgG1b, or IgG2) or a functional variant thereof. Immunoglobulins typically have long circulating half-lives in vivo. By fusing the GLP-1 receptor agonist (and leader) to an IgG Fc, the circulation time of the fusion protein is prolonged, while the function of the GLP-1 is preserved.

Two subclasses of the feline IgG constant domain are described, IgG1 and IgG2, with IgG1 being the predominant subclass (˜98%). Two alleles of the feline IGHG1 heavy chain gene (Cγ1a and Cγ1b) encode IgG heavy chain 1a and 1b proteins, and the usage frequency of each gene has been reported to be approximately 62% and 36%, respectively. Lu et al, Sequence analysis of feline immunoglobulin mRNAs and the development of a felinized monoclonal antibody specific to feline panleukopenia virus, Sci Rep. October 2017; 7: 12713, which is incorporated herein by reference.

As used herein, the Fc portion of an immunoglobulin has the meaning commonly given to the term in the field of immunology. Specifically, this term refers to an antibody fragment which does not contain the two antigen binding regions (the Fab fragments) from the antibody. The Fc portion consists of the constant region of an antibody from both heavy chains, which associate through non-covalent interactions and disulfide bonds. The Fc portion can include the hinge regions and extend through the CH2 and CH3 domains to the c-terminus of the antibody. The Fc portion can further include one or more glycosylation sites. In one embodiment, the fusion domain is a feline IgG Fc. The Fc domain can be derived from any feline IgG, including feline IgG1a, feline IgG1b, or feline IgG2. In one embodiment, the feline IgG Fc is SEQ ID NO: 11:

ARKTDHPPGPKPCDCPKCPPPEMLGGPSIFIFPPKPKDTLSISRTPEVTCLVVDL GPDDSDVQITWFVDNTQVYTAKTSPREEQFNSTYRVVSVLPILHQDWLKGKEFKCK VNSKSLPSPIERTISKAKGQPHEPQVYVLPPAQEELSRNKVSVTCLIKSFHPPDIAVEW EITGQPEPENNYRTTPPQLDSDGTYFVYSKLSVDRSHWQRGNTYTCSVSHEALHSHH TQKSLTQSPG. In another embodiment, the feline IgG Fc shares at least 90% identity, at least 95% identity, at least 99% identity, or at least 100% identity to SEQ ID NO: 11.

In another embodiment, the fusion domain is a feline albumin or a functional variant thereof. In one embodiment, the feline albumin is SEQ ID NO: 12: EAHQSEIAHRFNDLGEEHFRGLVLVAFSQYLQQCPFEDHVKLVNEVTEFAKGCVAD QSAANCEKSLHELLGDKLCTVASLRDKYGEMADCCEKKEPERNECFLQHKDDNPGF GQLVTPEADAMCTAFHENEQRFLGKYLYEIARRHPYFYAPELLYYAEEYKGVFTEC CEAADKAACLTPKVDALREKVLASSAKERLKCASLQKFGERAFKAWSVARLSQKFP KAEFAEISKLVTDLAKIHKECCHGDLLECADDRADLAKYICENQDSISTKLKECCGKP VLEKSHCISEVERDELPADLPPLAVDFVEDKEVCKNYQEAKDVFLGTFLYEYSRRHP EYSVSLLLRLAKEYEATLEKCCATDDPPACYAHVFDEFKPLVEEPHNLVKTNCELFE KLGEYGFQNALLVRYTKKVPQVSTPTLVEVSRSLGKVGSKCCTHPEAERLSCAEDYL SVVLNRLCVLHEKTPVSERVTKCCTESLVNRRPCFSALQVDETYVPKEFSAETFTFHA DLCTLPEAEKQIKKQSALVELLKHKPKATEEQLKTVMGDFGSFVDKCCAAEDKEAC FAEEGPKLVAAAQAALA. In another embodiment, the feline albumin shares at least 90% identity, at least 95% identity, at least 99% identity, or at least 100% identity to SEQ ID NO: 12.

The in vivo function and stability of the fusion proteins of the present disclosure may be optimized by adding small peptide linkers, e.g., to prevent potentially unwanted domain interactions or for other reasons. Further, a glycine-rich linker may provide some structural flexibility such that the GLP-1 analog portion can interact productively with the GLP-1 receptor on target cells such as the beta cells of the pancreas. Thus, the C-terminus of the GLP-1 analog and the N-terminus of the fusion domain of the fusion protein are, in one embodiment, fused via a linker. In one embodiment, the linker includes 1, 1.5 or 2 repeats of a G-rich peptide linker having the sequence GGGGSGGGGSGGGGS (SEQ ID NO: 13).

In one embodiment, the fusion protein comprises (a) feline thrombin leader, (b) a DPP-IV resistant variant of GLP-1(7-37), a linker, and (c) a feline IgG Fc. In one embodiment, the fusion protein has the sequence of SEQ ID NO: 14, or a sequence at least 90%, at least 95%, at least 98%, or at least 99% identical thereto.

SEQ ID NO: 14
MAHIRGLWLPGCLALAALCSLVHSQHVFLAPQQALSLLQRVRRHGEGTF
TSDVSSYLEEQAAKEFIAWLVKGGGGGGGSGGGGSGGGGSARKTDHPPG
PKPCDCPKCPPPEMLGGPSIFIFPPKPKDTLSISRTPEVTCLVVDLGPD
DSDVQITWFVDNTQVYTAKTSPREEQFNSTYRVVSVLPILHQDWLKGKE
FKCKVNSKSLPSPIERTISKAKGQPHEPQVYVLPPAQEELSRNKVSVTC
LIKSFHPPDIAVEWEITGQPEPENNYRTTPPQLDSDGTYFVYSKLSVDR
SHWQRGNTYTCSVSHEALHSHHTQKSLTQSPG

In one embodiment, the sequence encoding the fusion protein is SEQ ID NO: 15 or a sequence at least 75%, at least 85%, at least 90%, at least 95%, at least 98%, or at least 99% identical thereto.

SEQ ID NO: 15
atggctcacatcagaggactttggctgcctggctgtctggctctggctg
ctctgtgttctctggtgcacagccagcacgtgtttctggcccctcagca
ggctctgtccctgctgcaaagagttagaaggcacggcgagggcaccttc
acctccgacgtgtctagctacctggaagaacaggccgccaaagagttta
tcgcctggctggtcaaaggtggcggcggaggcggaggaagcggtggcgg
aggttcaggtggtggtggatctgccagaaagaccgaccatcctcctgga
cctaagccttgcgactgccctaagtgtcctccacctgagatgctcggcg
gacccagcatcttcatcttcccacctaagccaaaggacaccctgagcat
cagcagaacccctgaagtgacctgcctggtcgttgatctgggccccgac
gatagcgacgtgcagatcacttggtttgtggacaacacccaggtgtaca
cagccaagacaagccccagagaggaacagttcaacagcacctacagagt
ggtgtccgtgctgcccatcctgcaccaggattggctgaagggcaaagaa
ttcaagtgcaaagtgaacagcaagagcctgccttctccaatcgagcgga
ccatcagcaaggccaagggacagcctcacgagcctcaggtgtacgtcct
gcctcctgctcaagaggaactgagccggaacaaagtgtccgtgacctgt
ctgatcaagagctttcacccacctgatatcgccgtggaatgggagatca
caggccagcctgagcctgagaacaactaccggactacccctccacagct
ggactccgatggcacctacttcgtgtacagcaagctgagcgtggacaga
agccactggcagcggggcaatacctacacctgttccgtgtctcacgagg
ccctgcacagccaccacacacagaagtctctgacacagagccccggctg
a

In one embodiment, the fusion protein comprises (a) feline thrombin leader, (b) a DPP-IV resistant variant of GLP-1(7-37), a linker, and (c) a feline albumin. In one embodiment, the fusion protein has the sequence of SEQ ID NO: 16, or a sequence at least 90%, at least 95%, at least 98%, or at least 99% identical thereto.

SEQ ID NO: 16
MAHIRGLWLPGCLALAALCSLVHSQHVFLAPQQALSLLQRVRRHGEGTF
TSDVSSYLEEQAAKEFIAWLVKGGGGSGGGGSGGGASEAHQSEIAHRFN
DLGEEHFRGLVLVAFSQYLQQCPFEDHVKLVNEVTEFAKGCVADQSAAN
CEKSLHELLGDKLCTVASLRDKYGEMADCCEKKEPERNECFLQHKDDNP
GFGQLVTPEADAMCTAFHENEQRFLGKYLYELARRHPYFYAPELLYYAE
EYKGVFTECCEAADKAACLTPKVDALREKVLASSAKERLKCASLQKFGE
RAFKAWSVARLSQKFPKAEFAEISKLVTDLAKIHKECCHGDLLECADDR
ADLAKYICENQDSISTKLKECCGKPVLEKSHCISEVERDELPADLPPLA
VDFVEDKEVCKNYQEAKDVFLGTFLYEYSRRHPEYSVSLLLRLAKEYEA
TLEKCCATDDPPACYAHVFDEFKPLVEEPHNLVKTNCELFEKLGEYGFQ
NALLVRYTKKVPQVSTPTLVEVSRSLGKVGSKCCTHPEAERLSCAEDYL
SVVLNRLCVLHEKTPVSERVTKCCTESLVNRRPCFSALQVDETYVPKEF
SAETFTFHADLCTLPEAEKQIKKQSALVELLKHKPKATEEQLKTVMGDF
GSFVDKCCAAEDKEACFAEEGPKLVAAAQAALA.

In one embodiment, the sequence encoding the fusion protein is SEQ ID NO: 17 or a sequence at least 75%, at least 85%, at least 90%, at least 95%, at least 98%, or at least 99% identical thereto.

SEQ ID NO: 17
atggctcacatcagaggactttggctgcctggctgtctggctctggctg
ctctgtgttctctggtgcacagccagcacgtgtttctggcccctcagca
ggctctgtccctgctgcaaagagttagaaggcacggcgagggcaccttc
acctccgacgtgtccagctacctggaagaacaggccgccaaagagttta
tcgcctggctggtcaaaggcggcggaggatctggcggaggtggaagcgg
aggcggagcttctgaagcccaccagtctgagatcgcccaccggttcaac
gacctgggcgaagaacacttcagaggcctggtgctggtggcctttagcc
agtacctgcagcagtgccccttcgaggatcacgtgaagctggtcaacga
agtgaccgagttcgccaagggctgtgtggccgatcagtctgccgccaat
tgcgagaagtctctgcacgagctgctgggcgataagctgtgtacagtgg
ccagcctgcgggataagtacggcgagatggccgactgctgcgagaagaa
agagcccgagagaaacgagtgcttcctgcagcacaaggacgacaaccct
ggctttggccagctggttacacctgaggccgacgccatgtgcaccgcct
tccacgagaacgagcagagattcctgggcaagtacctgtacgagatcgc
cagacggcacccctacttttacgcccctgagctgctgtactacgccgaa
gagtacaagggcgtgttcaccgagtgttgcgaggccgctgataaggccg
cttgtctgacacccaaagtggacgccctgagagaaaaggtgctggcctc
cagcgccaaagaaagactgaagtgcgcctctctgcaaaagttcggcgag
agagccttcaaggcttggagcgtggcaagactgagccagaagttcccca
aggccgagtttgccgagatcagcaagctcgtgaccgacctggccaagat
ccacaaagagtgctgccacggcgacctgctggaatgcgctgacgataga
gccgatctggctaagtacatctgcgagaaccaggacagcatcagcacca
agctgaaagagtgctgcggcaagcccgtgctggaaaagagccactgtat
cagcgaggtggaacgggacgagctgcctgctgatctgcctcctctggcc
gtggacttcgtcgaggacaaagaagtgtgcaagaactaccaagaggcca
aggacgtgttcctgggaacctttctgtacgagtactctcggcggcaccc
cgagtattccgttagcctgctgctgcggctggcaaaagagtacgaggcc
acactggaaaagtgctgcgccaccgacgatcctccagcctgttacgccc
acgtgttcgacgagttcaagcccctggtggaagaaccccacaacctggt
caagaccaactgcgaactgttcgagaagctgggcgagtacggcttccag
aacgccctgctcgtgcggtacaccaagaaggtgccccaggtttccacac
ctacactggtcgaggtgtcccgctctctgggcaaagtgggcagcaagtg
ctgtacacacccagaggccgagagactgagctgcgccgaggattatctg
agcgtggtgctgaacagactgtgcgtgctgcacgagaaaacccctgtgt
ctgagcgcgtgaccaagtgttgtaccgagagcctggtcaacagacggcc
ttgctttagcgccctgcaagtcgacgagacatacgtgccaaaagagttc
agcgccgagacattcaccttccacgccgacctgtgtacactgcccgagg
ccgaaaagcagatcaagaaacagagcgccctggtcgaactgctgaagca
caagcctaaggccaccgaggaacagctgaaaaccgtgatgggcgacttc
ggcagcttcgtggataagtgctgtgccgctgaggacaaagaggcctgct
tcgctgaagagggccctaagttggttgccgctgctcaagctgccctggc
ctgataa

In another embodiment, the fusion protein comprises fusion protein comprises (a) feline thrombin leader, (b) two tandem copies of feline GLP-1(7-37) or a DPP-IV resistant variant thereof, a linker, and (c) a feline albumin. In one embodiment, the fusion protein has the sequence of SEQ ID NO: 18, or a sequence at least 90%, at least 95%, at least 98%, or at least 99% identical thereto.

SEQ ID NO: 18
MAHIRGLWLPGCLALAALCSLVHSQHVFLAPQQALSLLQRVRRHGEGTF
TSDVSSYLEGQAAKEFIAWLVKGRHGEGTFTSDVSSYLEGQAAKEFIAW
LVKGREAHQSEIAHRFNDLGEEHFRGLVLVAFSQYLQQCPFEDHVKLVN
EVTEFAKGCVADQSAANCEKSLHELLGDKLCTVASLRDKYGEMADCCEK
KEPERNECFLQHKDDNPGFGQLVTPEADAMCTAFHENEQRFLGKYLYEI
ARRHPYFYAPELLYYAEEYKGVFTECCEAADKAACLTPKVDALREKVLA
SSAKERLKCASLQKFGERAFKAWSVARLSQKFPKAEFAEISKLVTDLAK
IHKECCHGDLLECADDRADLAKYICENQDSISTKLKECCGKPVLEKSHC
ISEVERDELPADLPPLAVDFVEDKEVCKNYQEAKDVFLGTFLYEYSRRH
PEYSVSLLLRLAKEYEATLEKCCATDDPPACYAHVFDEFKPLVEEPHNL
VKTNCELFEKLGEYGFQNALLVRYTKKVPQVSTPTLVEVSRSLGKVGSK
CCTHPEAERLSCAEDYLSVVLNRLCVLHEKTPVSERVTKCCTESLVNRR
PCFSALQVDETYVPKEFSAETFTFHADLCTLPEAEKQIKKQSALVELLK
HKPKATEEQLKTVMGDFGSFVDKCCAAEDKEACFAEEGPKLVAAAQAAL
A

In one embodiment, the fusion protein has the sequence of SEQ ID NO: 20, or a sequence at least 90%, at least 95%, at least 98%, or at least 99% identical thereto.

SEQ ID NO: 20
MAHIRGLWLPGCLALAALCSLVHSQHVFLAPQQALSLLQRVRRHGEGTF
TSDVSSYLEGQAAKEFIAWLVKGREAHQSEIAHRFNDLGEEHFRGLVLV
AFSQYLQQCPFEDHVKLVNEVTEFAKGCVADQSAANCEKSLHELLGDKL
CTVASLRDKYGEMADCCEKKEPERNECFLQHKDDNPGFGQLVTPEADAM
CTAFHENEQRFLGKYLYEIARRHPYFYAPELLYYAEEYKGVFTECCEAA
DKAACLTPKVDALREKVLASSAKERLKCASLQKFGERAFKAWSVARLSQ
KFPKAEFAEISKLVTDLAKIHKECCHGDLLECADDRADLAKYICENQDS
ISTKLKECCGKPVLEKSHCISEVERDELPADLPPLAVDFVEDKEVCKNY
QEAKDVFLGTFLYEYSRRHPEYSVSLLLRLAKEYEATLEKCCATDDPPA
CYAHVFDEFKPLVEEPHNLVKTNCELFEKLGEYGFQNALLVRYTKKVPQ
VSTPTLVEVSRSLGKVGSKCCTHPEAERLSCAEDYLSVVLNRLCVLHEK
TPVSERVTKCCTESLVNRRPCFSALQVDETYVPKEFSAETFTFHADLCT
LPEAEKQIKKQSALVELLKHKPKATEEQLKTVMGDFGSFVDKCCAAEDK
EACFAEEGPKLVAAAQAALA

In one embodiment, the sequence encoding the fusion protein is SEQ ID NO: 19 or a sequence at least 75%, at least 85%, at least 90%, at least 95%, at least 98%, or at least 99% identical thereto.

SEQ ID NO: 19
atggctcacatcagaggactttggctgcctggctgtctggctctggctg
ctctgtgttctctggtgcacagccagcacgtgtttctggcccctcagca
ggctctgtccctgctgcaaagagttagaaggcacggcgagggcaccttc
acctccgacgtgtctagctacctggaaggacaggccgccaaagagttta
tcgcctggctggtcaaaggcagacacggcgaagggacattcacaagcga
cgtgtcctcttatctcgaaggccaggctgctaaagagttcattgcttgg
ctcgtgaagggcagagaggcccaccagtctgagatcgcccacagattca
acgacctgggcgaagaacacttcagaggcctggtgctggtggccttcag
ccagtatctgcagcagtgccccttcgaggaccacgtgaagctggtcaac
gaagtgaccgagttcgccaagggctgtgtggccgatcagtctgccgcca
attgcgagaagtctctgcacgagctgctgggcgataagctgtgtacagt
ggccagcctgcgggataagtacggcgagatggccgactgctgcgagaag
aaagagcccgagagaaacgagtgcttcctgcagcacaaggacgacaacc
ctggctttggccagctggttacacctgaggccgacgccatgtgcaccgc
cttccacgagaacgagcagagattcctgggcaagtacctgtacgagatc
gccagacggcacccctacttttacgcccctgagctgctgtactacgccg
aagagtacaagggcgtgttcaccgagtgttgcgaggccgctgataaggc
cgcttgtctgacacccaaagtggacgccctgagagaaaaggtgctggcc
tccagcgccaaagaaagactgaagtgcgcctctctgcaaaagttcggcg
agagagccttcaaggcttggagcgtggcaagactgagccagaagttccc
caaggccgagtttgccgagatcagcaagctcgtgaccgacctggccaag
atccacaaagagtgctgccacggcgacctgctggaatgcgctgacgata
gagccgatctggctaagtacatctgcgagaaccaggacagcatcagcac
caagctgaaagagtgctgcggcaagcccgtgctggaaaagagccactgt
atcagcgaggtggaacgggacgagctgcctgctgatctgcctcctctgg
ccgtggacttcgtcgaggacaaagaagtgtgcaagaactaccaagaggc
caaggacgtgttcctgggaacctttctgtacgagtactctcggcggcac
cccgagtattccgttagcctgctgctgcggctggcaaaagagtacgagg
ccacactggaaaagtgctgcgccaccgacgatcctccagcctgttacgc
ccacgtgttcgacgagttcaagcccctggtggaagaaccccacaacctg
gtcaagaccaactgcgaactgttcgagaagctgggcgagtacggcttcc
agaacgccctgctcgtgcggtacaccaagaaggtgccccaggtttccac
acctacactggtcgaggtgtcccgctctctgggcaaagtgggcagcaag
tgctgtacacacccagaggccgagagactgagctgcgccgaggattatc
tgagcgtggtgctgaacagactgtgcgtgctgcacgagaaaacccctgt
gtctgagcgcgtgaccaagtgttgtaccgagagcctggtcaacagacgg
ccttgctttagcgccctgcaagtcgacgagacatacgtgccaaaagagt
tcagcgccgagacattcaccttccacgccgacctgtgtacactgcccga
ggccgaaaagcagatcaagaaacagagcgccctggtcgaactgctgaag
cacaagcctaaggccaccgaggaacagctgaaaaccgtgatgggcgact
tcggcagcttcgtggataagtgctgtgccgctgaggacaaagaggcctg
cttcgctgaagagggccctaagttggttgccgctgctcaagctgccctg
gcctgataa

When a variant or fragment of the leader sequence, GLP-1 receptor agonist, or fusion domain is desired, the coding sequences for these peptides may be generated using site-directed mutagenesis of the wild-type nucleic acid sequence. Alternatively or additionally, web-based or commercially available computer programs, as well as service based companies may be used to back translate the amino acids sequences to nucleic acid coding sequences, including both RNA and/or cDNA. See, e.g., backtranseq by EMBOSS, ebi.ac.uk/Tools/st/; Gene Infinity (geneinfinity.org/sms-/sms_backtranslation.html); ExPasy (expasy.org/tools/). In one embodiment, the RNA and/or cDNA coding sequences are designed for optimal expression in the subject species for which administration is ultimately intended, i.e., a feline.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In addition to the leader sequences, GLP-1 receptor agonists, fusion domains, and fusion proteins provided herein, nucleic acid sequences encoding these polypeptides are provided. In one embodiment, a nucleic acid sequence is provided which encodes for the GLP-1 peptides described herein. In some embodiments, this may include any nucleic acid sequence which encodes the GLP-1 sequence of SEQ ID NO: 1. In another embodiment, this includes any nucleic acid which includes the GLP-1 sequence of SEQ ID NO: 2. In another embodiment, this includes any nucleic acid which includes the GLP-1 sequence of SEQ ID NO: 3. In another embodiment, this includes any nucleic acid which includes the GLP-1 sequence of SEQ ID NO: 4. In another embodiment, this includes any nucleic acid which includes the GLP-1 sequence of SEQ ID NO: 5. In another embodiment, this includes any nucleic acid which includes the GLP-1 sequence of SEQ ID NO: 6.

In one embodiment, a nucleic acid sequence is provided which encodes for the GLP-1 fusion protein described herein. In another embodiment, this includes any nucleic acid sequence which encodes the GLP-1 fusion protein of SEQ ID NO: 14. In another embodiment, this includes any nucleic acid sequence which encodes the GLP-1 fusion protein of SEQ ID NO: 16. In another embodiment, this includes any nucleic acid sequence which encodes the GLP-1 fusion protein of SEQ ID NO: 18. In another embodiment, this includes any nucleic acid sequence which encodes the GLP-1 fusion protein of SEQ ID NO: 20.

In certain embodiments of the viral vectors described herein, the viral vector is an adeno-associated virus (AAV) viral vector or recombinant AAV (rAAV). The term “recombinant AAV” or “rAAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged an expression cassette flanked by AAV inverted terminal repeat sequences (ITRs) (together referred to as the “vector genome”) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAVrh91 capsid or variant thereof. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAVAnc80, AAV10, AAV11, AAV12, AAVrh8, AAVrh74, AAV-DJ8, AAV-DJ, AAVhu.37, AAVrh.64R1, and AAVhu68. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh10), WO 2005/033321, WO 2018/160582 (AAVhu68), which are incorporated herein by reference. Other suitable AAVs may include, without limitation, AAVrh90 [PCT/US20/30273, filed Apr. 28, 2020], AAVrh91 [PCT/US20/030266, filed Apr. 28, 2020, now a publication WO 2020/223231, published Nov. 5, 2020], AAVrh92, AAVrh93, AAVrh91.93 [PCT/US20/30281, filed Apr. 28, 2020], which are incorporated by reference herein. Other suitable AAV include AAV3B variants which are described in U.S. Provisional Patent Application No. 62/924,112, filed Oct. 21, 2019, and U.S. Provisional Patent Application No. 63/025,753, filed May 15, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. See also, International Patent Application No. PCT/US21/45945, filed Aug. 13, 2021, U.S. Provisional Patent Application No. 63/065,616, filed Aug. 14, 2020, and U.S. Provisional Patent Application No. 63/109,734, filed Nov. 4, 2020, which are all incorporated herein by reference in its entireties. These documents also describe other AAV capsids which may be selected for generating rAAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

In one embodiment, the viral vector is an rAAV having the capsid of AAV8 or a functional variant thereof. In one embodiment, the viral vector is an rAAV having the capsid of AAVrh91 or a functional variant thereof. In one embodiment, the viral vector is an rAAV having the capsid of AAV3.AR.2.12 or a functional variant thereof. In one embodiment, the viral vector is an rAAV having a capsid selected from AAV9, AAVrh64R1, AAVhu37, or AAVrh10.

In certain embodiments, a novel isolated AAVrh91 capsid is provided. A nucleic acid sequence encoding the AAVrh91 capsid is provided in SEQ ID NO: 24 and the encoded amino acid sequence is provided in SEQ ID NO: 26. Provided herein is an rAAV comprising at least one of the vp1, vp2 and the vp3 of AAVrh91 (SEQ ID NO: 26). Also provided herein are rAAV comprising an AAV capsid encoded by at least one of the vp1, vp2 and the vp3 of AAVrh91 (SEQ ID NO: 24). In yet another embodiment, a nucleic acid sequence encoding the AAVrh91 amino acid sequence is provided in SEQ ID NO: 24 and the encoded amino acid sequence is provided in SEQ ID NO: 26. Also provided herein are rAAV comprising an AAV capsid encoded by at least one of the vp1, vp2 and the vp3 of AAVrh91eng (SEQ ID NO: 25). In certain embodiments, the vp1, vp2 and/or vp3 is the full-length capsid protein of AAVrh91 (SEQ ID NO: 26). In other embodiments, the vp1, vp2 and/or vp3 has an N-terminal and/or a C-terminal truncation (e.g., truncation(s) of about 1 to about 10 amino acids).

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 26, vp1 proteins produced from SEQ ID NO: 24, or vp1 proteins produced from a nucleic acid sequence at least 70% identical to SEQ ID NO: 24 which encodes the predicted amino acid sequence of 1 to 736 of SEQ ID NO: 26, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 26, vp2 proteins produced from a sequence comprising at least nucleotides 412 to 2208 of SEQ ID NO: 24, or vp2 proteins produced from a nucleic acid sequence at least 70% identical to at least nucleotides 412 to 2208 of SEQ ID NO: 24 which encodes the predicted amino acid sequence of at least about amino acids 138 to 736 of SEQ ID NO: 26, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 26, vp3 proteins produced from a sequence comprising at least nucleotides 607 to 2208 of SEQ ID NO: 24, or vp3 proteins produced from a nucleic acid sequence at least 70% identical to at least nucleotides 607 to 2208 of SEQ ID NO: 24 which encodes the predicted amino acid sequence of at least about amino acids 203 to 736 of SEQ ID NO: 26; and/or (2) a heterogeneous population of vp1 proteins which are the product of a nucleic acid sequence encoding the amino acid sequence of SEQ ID NO: 26, a heterogeneous population of vp2 proteins which are the product of a nucleic acid sequence encoding the amino acid sequence of at least about amino acids 138 to 736 of SEQ ID NO: 26, and a heterogeneous population of vp3 proteins which are the product of a nucleic acid sequence encoding at least amino acids 203 to 736 of SEQ ID NO: 26, wherein: the vp1, vp2 and vp3 proteins contain subpopulations with amino acid modifications comprising at least two highly deamidated asparagines (N) in asparagine-glycine pairs in SEQ ID NO: 26 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 26, vp1 proteins produced from SEQ ID NO: 25, or vp1 proteins produced from a nucleic acid sequence at least 70% identical to SEQ ID NO: 25 which encodes the predicted amino acid sequence of 1 to 736 of SEQ ID NO: 26, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 26, vp2 proteins produced from a sequence comprising at least nucleotides 412 to 2208 of SEQ ID NO: 25, or vp2 proteins produced from a nucleic acid sequence at least 70% identical to at least nucleotides 412 to 2208 of SEQ ID NO: 25 which encodes the predicted amino acid sequence of at least about amino acids 138 to 736 of SEQ ID NO: 26, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 26, vp3 proteins produced from a sequence comprising at least nucleotides 607 to 2208 of SEQ ID NO: 25, or vp3 proteins produced from a nucleic acid sequence at least 70% identical to at least nucleotides 607 to 2208 of SEQ ID NO: 25 which encodes the predicted amino acid sequence of at least about amino acids 203 to 736 of SEQ ID NO: 26; and/or (2) a heterogeneous population of vp1 proteins which are the product of a nucleic acid sequence encoding the amino acid sequence of SEQ ID NO: 26, a heterogeneous population of vp2 proteins which are the product of a nucleic acid sequence encoding the amino acid sequence of at least about amino acids 138 to 736 of SEQ ID NO: 26, and a heterogeneous population of vp3 proteins which are the product of a nucleic acid sequence encoding at least amino acids 203 to 736 of SEQ ID NO: 26, wherein: the vp1, vp2 and vp3 proteins contain subpopulations with amino acid modifications comprising at least two highly deamidated asparagines (N) in asparagine-glycine pairs in SEQ ID NO: 26 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the AAVrh91 vp1, vp2 and vp3 proteins contain subpopulations with amino acid modifications comprising at least two highly deamidated asparagines (N) in asparagine-glycine pairs in SEQ ID NO: 26 and optionally further comprising subpopulations comprising other deamidated amino acids, wherein the deamidation results in an amino acid change. High levels of deamidation at N-G pairs N57, N383 and/or N512 are observed, relative to the number of SEQ ID NO: 26. Deamidation has been observed in other residues. In certain embodiments, AAVrh91 may have other residues deamidated, e.g., typically at less than 10% and/or may have other modifications, including phosphorylation (e.g., where present, in the range of about 2 to about 30%, or about 2 to about 20%, or about 2 to about 10%) (e.g., at S149), or oxidation (e.g, at one or more of ˜W22, ˜M211, W247, M403, M435, M471, W478, W503, ˜M537, ˜M541, ˜M559, ˜M599, M635, and/or, W695). Optionally the W may oxidize to kynurenine.

TABLE A
AAVrh91 Deamidation

| AAVrh91 | |
|---|---|
| Deamidation based | |
| on VP1 numbering | % Deamidation |
| N57 + Deamidation | 65-90, 70-95, 80-95, 75-100, 80-100, or 90-100 |
| N94 + Deamidation | 2-15 or 2-5 |
| N303 + Deamidation | 2-15 or 5-10 |
| N383 + Deamidation | 65-90, 70-95, 80-95, 75-100, 80-100, or 90-100 |
| N497 + Deamidation | 2-15 or 5-10 |
| N512 + Deamidation | 65-90, 70-95, 80-95, 75-100, 80-100, or 90-100 |
| ~N691 + Deamidation | 2-15, 2-10, or 5-10 |

In certain embodiments, an AAVrh91 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh91 sequence provided herein. See, SEQ ID NO: 26.

In certain embodiments, an AAVrh91 capsid comprises: a heterogeneous population of vp1 proteins which are the product of a nucleic acid sequence encoding the amino acid sequence of SEQ ID NO: 26, a heterogeneous population of vp2 proteins which are the product of a nucleic acid sequence encoding the amino acid sequence of at least about amino acids 138 to 736 of SEQ ID NO: 26, and a heterogeneous population of vp3 proteins which are the product of a nucleic acid sequence encoding at least amino acids 203 to 736 of SEQ ID NO: 26.

In certain embodiments, the modified AAVrh91 nucleic acid sequences is be used to generate a mutant rAAV having a capsid with lower deamidation than the native AAVrh91 capsid. Such mutant rAAV may have reduced immunogenicity and/or increase stability on storage, particularly storage in suspension form.

In one aspect, a recombinant AAV (rAAV) is provided. The rAAV includes an AAV capsid from adeno-associated virus rh91, and a vector genome packaged in the AAV capsid, said vector genome comprising AAV inverted terminal repeats (ITRs), a coding sequence for the feline GLP-1 receptor agonist of SEQ ID NO: 14, and regulatory sequences which direct expression of the feline GLP-1 receptor agonist.

In one embodiment, the rAAV is an scAAV. The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In one embodiment, the nucleic acid sequences encoding the GLP-1 constructs described herein are engineered into any suitable genetic element, e.g., naked DNA, phage, transposon, cosmid, RNA molecule (e.g., mRNA), episome, etc., which transfers the GLP-1 sequences carried thereon to a host cell, e.g., for generating nanoparticles carrying DNA or RNA, viral vectors in a packaging host cell and/or for delivery to a host cell in a subject. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, the term “host cell” may refer to the packaging cell line in which a vector (e.g., a recombinant AAV or rAAV) is produced from a production plasmid. In the alternative, the term “host cell” may refer to any target cell in which expression of a gene product described herein is desired. Thus, a “host cell,” refers to a prokaryotic or eukaryotic cell (e.g., bacterial cell, human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. In a further embodiment, the term “host cell” is an intestine cell, a small intestine cell, a pancreatic cell, a liver cell.

As used herein, the term “target cell” refers to any target cell in which expression of a heterologous nucleic acid sequence or protein is desired. In certain embodiments, the target cell is a liver cell. In other embodiments, the target cell is a muscle cell.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences (also referred to as elements) that are contiguous or non-contiguous with the nucleic acid sequence and regulatory sequences that act in trans or cis nucleic acid sequence. Such regulatory sequences typically include, e.g., one or more of a promoter, an enhancer, a transcription factor, transcription terminator, an intron, sequences that enhance translation efficiency (i.e., a Kozak consensus sequence), efficient RNA processing signals such as slicing and a polyadenylation sequence, sequences that stabilize cytoplasmic mRNA, for example Woodchuck Hepatitis Virus (WHP) posttranslational Regulatory Element (WPRE), and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

In one embodiment, the expression cassette refers to a nucleic acid molecule which comprises the GLP-1 construct coding sequences (e.g., coding sequences for the GLP-1 fusion protein), promoter, and may include other regulatory sequences therefor, which cassette may be engineered into a genetic element and/or packaged into the capsid of a viral vector (e.g., a viral particle). Typically, such an expression cassette for generating a viral vector contains the GLP-1 construct sequences described herein flanked by packaging signals of the viral genome and other expression control sequences such as those described herein. Any of the expression control sequences can be optimized for a specific species using techniques known in the art including, e.g., codon optimization, as described herein.

The expression cassette typically contains a promoter sequence as part of the expression control sequences. In one embodiment, the liver-specific promoter thyroxin binding globulin (TBG) is used. In the plasmids and vectors described herein, a CB7 promoter is used. CB7 is a chicken R-actin promoter with cytomegalovirus enhancer elements. Alternatively, other liver-specific promoters may be used, such as those listed in the The Liver Specific Gene Promoter Database, Cold Spring Harbor, rulai.schl.edu/LSPD, and including but not limited to alpha 1 anti-trypsin (AlAT); human albumin (Miyatake et al., J. Virol. 71:5124 32 (1997)), humAlb; hepatitis B virus core promoter (Sandig et al., Gene Ther. 3:1002 9 (1996)); Or a TTR minimal enhancer/promoter, alpha-antitrypsin promoter, or liver-specific promoter (LSP) (Wu et al. Mol Ther. 16:280-289 (2008)). Other promoters, such as viral promoters, constitutive promoters, regulatable promoters (see, e.g., WO 2011/126808 and WO 2013/04943) or a promoter responsive to physiologic cues may be used may be utilized in the vectors described herein.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), human growth hormone (hGH), SV40, rabbit β-globin (also referred to as rabbit globin polyA; RGB), modified RGB (mRGB), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. In one embodiment, the polyA is a rabbit globin polyA.

These control sequences are “operably linked” to the GLP-1 construct sequences. As used herein, the term “operably linked” refers to both expression control sequences that are contiguous with the gene of interest and expression control sequences that act in trans or at a distance to control the gene of interest.

In one embodiment, a rAAV is provided which includes a 5′ ITR, CB7 promoter, chicken beta-actin intron, coding sequence for the fusion protein of SEQ ID NO: 14, a rabbit globin poly A, and a 3′ ITR. In one embodiment, a rAAV is provided which includes a 5′ ITR, CB7 promoter, chicken beta-actin intron, coding sequence for the fusion protein of SEQ ID NO: 16, a rabbit globin poly A, and a 3′ ITR. In one embodiment, a rAAV is provided which includes a 5′ ITR, CB7 promoter, chicken beta-actin intron, coding sequence for the fusion protein of SEQ ID NO: 18, a rabbit globin poly A, and a 3′ ITR. In one embodiment, a rAAV is provided which includes a 5′ ITR, CB7 promoter, chicken beta-actin intron, coding sequence for the fusion protein of SEQ ID NO: 20, a rabbit globin poly A, and a 3′ ITR.

The minimal sequences required to package the expression cassette into an AAV viral particle are the AAV 5′ and 3′ ITRs, which may be of the same AAV origin as the capsid, or of a different AAV origin (to produce an AAV pseudotype). In one embodiment, the ITR sequences from AAV2, or the deleted version thereof (AITR), are used for convenience and to accelerate regulatory approval. However, ITRs from other AAV sources may be selected. Preferably, the source of the ITRs is the same as the source of the Rep protein, which is provided in trans for production. Typically, an expression cassette for an AAV vector comprises an AAV 5′ ITR, the GLP-1 fusion protein coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. In one embodiment, a chimeric AAV capsid may be utilized. Still other AAV components may be selected. Sources of such AAV sequences are described herein and may also be isolated or obtained from academic, commercial, or public sources (e.g., the American Type Culture Collection, Manassas, VA). The AAV sequences may be obtained through synthetic or other suitable means by reference to published sequences such as are available in the literature or in databases such as, e.g., GenBank®, PubMed®, or the like.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

The rAAV described herein comprise a selected capsid with a vector genome packaged inside. The vector genome (or rAAV genome) comprises 5′ and 3′ AAV inverted terminal repeats (ITRs), the polynucleotide sequence encoding the fusion protein, and regulatory sequences which direct insertion of the polynucleotide sequence encoding the fusion protein to the genome of a host cell. In one embodiment, the vector genome is the sequence shown in SEQ ID NO: 21 or a sequence sharing at least 90%, at least 95%, or at least 99% identity therewith. In one embodiment, the vector genome is the sequence shown in SEQ ID NO: 22 or a sequence sharing at least 90%, at least 95%, or at least 99% identity therewith. In one embodiment, the vector genome is the sequence shown in SEQ ID NO: 23 or a sequence sharing at least 90%, at least 95%, or at least 99% identity therewith. In one embodiment, an expression cassette is provided having the sequence of nt 199 to 3125 of SEQ ID NO: 21 or a sequence sharing at least 90%, at least 95%, or at least 99% identity therewith. In one embodiment, an expression cassette is provided having the sequence of nt 199 to 4194 of SEQ ID NO: 22 or a sequence sharing at least 90%, at least 95%, or at least 99% identity therewith. In one embodiment, an expression cassette is provided having the sequence of nt 199 to 4143 of SEQ ID NO: 23 or a sequence sharing at least 90%, at least 95%, or at least 99% identity therewith.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding GLP-1 constructs operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

Optionally, the GLP-1 constructs described herein may be delivered via viral vectors other than rAAV. Such other viral vectors may include any virus suitable for gene therapy may be used, including but not limited to adenovirus; herpes virus; lentivirus; retrovirus; etc. Suitably, where one of these other vectors is generated, it is produced as a replication-defective viral vector.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Also provided are compositions which include the viral vector constructs described herein. The pharmaceutical compositions described herein are designed for delivery to feline subjects in need thereof by any suitable route or a combination of different routes. Direct delivery to the liver (optionally via intravenous, via the hepatic artery, or by transplant), oral, inhalation, intranasal, intratracheal, intraarterial, intraocular, intravenous, intramuscular, subcutaneous, intradermal, and other parental routes of administration. The viral vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus). In one embodiment, administration is intramuscular. In another embodiment, administration is intravenous.

The replication-defective viruses can be formulated with a physiologically acceptable carrier for use in gene transfer and gene therapy applications. In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies are the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

Also, the replication-defective virus compositions can be formulated in dosage units to contain an amount of replication-defective virus that is in the range of about 1.0×10⁹ GC to about 1.0×10¹⁵ GC. In another embodiment, this amount of viral genome may be delivered in split doses. In one embodiment, the dose is about 1.0×10¹⁰ GC to about 3.0×10¹³ GC for an average feline subject of about 5-10 kg. In another embodiment, the dose about 1×10⁹ GC. For example, the dose of AAV virus may be about 1×10¹⁰ GC, 1×10¹¹ GC, about 5×10¹¹ GC, about 1×10¹² GC, about 5×10¹² GC, or about 1×10¹³ GC. In another embodiment, the dosage is about 1.0×10⁹ GC/kg to about 3.0×10¹³ GC/kg for a feline subject. In another embodiment, the dose about 1×10⁹ GC/kg. For example, the dose of AAV virus may be about 1×10¹⁰ GC/kg, 1×10¹¹ GC/kg, about 5×10¹¹ GC/kg, about 1×10¹² GC/kg, about 5×10¹² GC/kg, or about 1×10¹³ GC/kg. In one embodiment, the constructs may be delivered in volumes from 1 μL to about 100 mL for a veterinary subject. See, e.g., Diehl et al, J. Applied Toxicology, 21:15-23 (2001) for a discussion of good practices for administration of substances to various veterinary animals. This document is incorporated herein by reference. As used herein, the term “dosage” or “amount” can refer to the total dosage or amount delivered to the subject in the course of treatment, or the amount delivered in a single unit (or multiple unit or split dosage) administration.

The above-described recombinant vectors may be delivered to host cells according to published methods. The rAAV, preferably suspended in a physiologically compatible carrier, may be administered to a desired subject including a feline. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

In another embodiment, the composition includes a carrier, diluent, excipient and/or adjuvant.

In certain embodiments, for administration to a human patient, the rAAV is suitably suspended in an aqueous solution containing saline, a surfactant, and a pharmaceutically and/or physiologically compatible salt or mixture of salts. Suitably, the formulation is adjusted to a physiologically acceptable pH, e.g., in the range of pH 6 to 9, or pH 6.0 to 7.5, or pH 6.2 to 7.7, or pH 6.5 to 7.5, pH 7.0 to 7.7, or pH 7.2 to 7.8, or about 7.0. In certain embodiments, the formulation is adjusted to a pH of about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8. In certain embodiments, a pH of about 7.28 to about 7.32, about 6.0 to about 7.5, about 6.2 to about 7.7, about 7.5 to about 7.8, about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8 may be desired. In certain embodiments, for intravenous delivery, a pH of about 6.8 to about 7.2 may be desired. However, other pHs within the broadest ranges and these subranges may be selected for other route of delivery.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells or target cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.0010% of the suspension.

Dosages of the vector depends primarily on factors such as the condition being treated, the age, weight and health of the feline patient, and may thus vary among patients. For example, a therapeutically effective feline dosage of viral vector is generally in the range of from about 25 to about 1000 microliters to about 100 mL of solution containing concentrations of from about 1×10⁹ to 1×10¹⁶ genomes virus vector (to treat average feline subject of 4.5 kg), including all integers or fractional amounts within the range. In certain embodiments, the feline patients are administered about 1×10⁹ GC/cat to about 1×10¹² GC/cat, or about 1×10¹⁰ GC/cat to about 1×10¹¹ GC/cat, including all integers or fractional amounts within the range.

The composition of the invention may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

In some embodiments, a concentration of a recombinant adeno-associated virus carrying a nucleic acid sequence encoding the desired transgene under the control of the regulatory sequences desirably ranges from about 10⁷ and 10¹⁴ vector genomes per milliliter (vg/mL) (also called genome copies/mL (GC/mL)) in a composition.

In one embodiment, the dosage of rAAV in a composition is from about 1.0×10⁹ GC/kg of body weight to about 3.0×10¹³ GC/kg. In one embodiment, the dosage is about 1×10¹¹ GC/kg. In one embodiment, the dosage is about 1.0×10¹³ GC/kg. In one embodiment, the dosage is about 1.0×10¹² GC/kg. In one embodiment, the dosage is about 5.0×10¹² GC/kg. All ranges described herein are inclusive of the endpoints.

In one embodiment, the effective dosage (total genome copies delivered) is from about 10⁷ to 10¹³ vector genomes. In one embodiment, the total dosage is about 108 genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed.

The viral vectors and other constructs described herein may be used in preparing a medicament for delivering a GLP-1 fusion protein construct to a subject in need thereof, supplying GLP-1 having an increased half-life to a subject, and/or for treating type I diabetes, type II diabetes or metabolic syndrome in a subject. Thus, in another aspect a method of treating diabetes is provided. The method includes administering a composition as described herein to a feline subject in need thereof. In one embodiment, the composition includes a viral vector containing a GLP-1 fusion protein expression cassette, as described herein.

As used herein, the term “treatment” or “treating” is defined encompassing administering to a subject one or more compounds or compositions described herein for the purposes of amelioration of one or more symptoms of type I diabetes, type II diabetes (T2DM) or metabolic syndrome. “Treatment” can thus include one or more of reducing progression of type I diabetes, type II diabetes or metabolic syndrome, reducing the severity of the symptoms, removing the disease symptoms, delaying progression of disease, or increasing efficacy of therapy in a given subject.

As used herein, the term “remission” refers to the ability to cease insulin treatment when the cat no longer exhibits clinical signs of diabetes and has normal blood glucose levels.

In another embodiment, a method for treating T2DM in a feline is provided. The method includes administering a viral vector comprising a nucleic acid molecule comprising a sequence encoding a fusion protein as described herein.

In another aspect, a method of treating a metabolic disease in a feline is provided. The method includes administering a composition as described herein to a feline subject in need thereof. In one embodiment, the composition includes a viral vector containing a GLP-1 fusion protein expression cassette, as described herein. In one embodiment, the metabolic disease is Type I diabetes. In one embodiment, the metabolic disease is Type II diabetes. In one embodiment, the metabolic disease is metabolic syndrome.

In another aspect a method of reducing body weight in a feline subject is provided. The method includes administering a composition as described herein to a subject in need thereof. In one embodiment, the composition includes a viral vector containing a GLP-1 fusion protein expression cassette, as described herein.

A course of treatment may optionally involve repeat administration of the same viral vector (e.g., an AAVrh91 vector) or a different viral vector (e.g., an AAVrh91 and an AAV3B.AR2.12). Still other combinations may be selected using the viral vectors described herein. Optionally, the composition described herein may be combined in a regimen involving other diabetic drugs or protein-based therapies (including e.g., GLP-1 analogues, insulin, oral antihyperglycemic drugs (sulfonylureas, biguanides, thiazolidinediones, and alpha-glucoidase inhibitors). Optionally, the composition described herein may be combined in a regimen involving lifestyle changes including dietary and exercise regimens. In certain embodiments, the AAV vector and the combination therapy are administered essentially simultaneously. In other embodiments, the AAV vector is administered first. In other embodiments, the combination therapy is delivered first.

In one embodiment, the composition is administered in combination with an effective amount of insulin. Various commercially available insulin products are known in the art, including, without limitation, protamine zinc recombinant human insulin (ProZinc®), porcine insulin zinc suspension (Vetsulin®), and insulin glargine (Lantus®). In some embodiments, combination of the rAAV described herein with insulin decreases insulin dose requirements in the subject, as compared to prior to treatment with the viral vector. Such dose requirements may be reduced by 10% or more, 20% or more, 30% or more, 40% or more, 50% or more, 60% or more, 70% or more, 80% or more, or 90% or more. The treating physician may determine the correct dosage of insulin needed by the subject. For example, the subject may be being treated using insulin or other therapy, which the treating physician may continue upon administration of the AAV vector. Such insulin or other co-therapy may be continued, reduced, or discontinued as needed subsequently.

In one embodiment, composition comprising the expression cassette, vector genome, rAAV, or other composition described herein for gene therapy is delivered as a single dose per patient. In one embodiment, the subject is delivered a therapeutically effective amount of a composition described herein. As used herein, a “therapeutically effective amount” refers to the amount of the expression cassette or vector, or a combination thereof that delivers and expresses in the target cells an amount of GLP1-Fc sufficient to reach therapeutic goal. In certain embodiments, the therapeutic goal is to ameliorate or treat one or more of the symptoms of type I diabetes, type II diabetes or metabolic syndrome. A therapeutically effective amount may be determined based on an animal model, rather than a feline patient. In another embodiment, the therapeutic goal is remission of the metabolic disease in the subject.

In certain embodiments, the effective dosage and/or the method results in expression of the fusion protein in the serum of the subject for at least three months, at least six months, or at least twelve months. In certain embodiments, the effective dosage and/or method results in expression of the fusion protein in the subject at a serum concentration of at least 3,000 picomolar (pM), at least 5,000 pM, at least 10,000 pm, at least 25,000 pM, or at least 50,000 pM for at least three months, at least six months, or at least twelve months. In other embodiments, the effective dosage and/or method results in expression of the fusion protein in the subject at a serum concentration of 3,000 picomolar (pM) to 200,000 pM, 5,000 picomolar (pM) to 200,000 pM, 10,000 picomolar (pM) to 200,000 pM, 25,000 picomolar (pM) to 200,000 pM, or 50,000 picomolar (pM) to 200,000 pM, for 3-12 months, 6-12 months, or twelve months. In certain embodiments, the effective dosage and/or the method results in expression of the fusion protein in the subject at a therapeutically effective concentration for at least three months, at least six months, or at least twelve months.

In other embodiments, a therapeutic goal is reduction of serum fructosamine. In certain embodiments, the effective amount and/or method is effective to decrease serum fructosamine in the subject by about 6%. In another embodiment, the effective amount and/or method is effective to decrease serum fructosamine in the subject by 5%-10%. In yet another embodiment, the effective amount and/or method is effective to decrease serum fructosamine in the subject by about 10%. In another embodiments, the effective amount and/or method is effective to decrease serum fructosamine in the subject by 10% to 20%. Other ranges and integers within the recited ranges are contemplated. As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 20 provides the encoded amino acid sequence of the AAVrh91 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

As used herein, a “stock” of rAAV refers to a population of rAAV. Despite heterogeneity in their capsid proteins due to deamidation, rAAV in a stock are expected to 5 share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein the terms “GLP-1 construct”, “GLP-1 expression construct” and synonyms include the GLP-1 sequence as described herein in combination with a leader and fusion domain. The terms “GLP-1 construct”, “GLP-1 expression construct” and synonyms can be used to refer to the nucleic acid sequences encoding the GLP-1 fusion protein or the expression products thereof.

The terms “percent (%) identity”, “sequence identity”, “percent sequence identity”, or “percent identical” in the context of nucleic acid sequences refers to the bases in the two sequences which are the same when aligned for correspondence. The length of sequence identity comparison may be over the full-length of the genome, the full-length of a gene coding sequence, or a fragment of at least about 100 to 150 nucleotides, or as desired. However, identity among smaller fragments, e.g., of at least about nine nucleotides, usually at least about 20 to 24 nucleotides, at least about 28 to 32 nucleotides, at least about 36 or more nucleotides, may also be desired. Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

By the term “highly conserved” is meant at least 80% identity, preferably at least 90% identity, and more preferably, over 97% identity. Identity is readily determined by one of skill in the art by resort to algorithms and computer programs known by those of skill in the art.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example, “95% identity” and “at least 95% identity” may be used interchangeably and include 95, 96, 97, 98, 99 up to 100% identity to the referenced sequence, and all fractions therebetween.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequencers. A suitable amino acid fragment may be at least about 8 amino acids in length, and may be up to about 150 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%, e.g., ±1, ±2, ±3, ±4, ±5, ±6, ±7, ±8, ±9, ±10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

The term “regulation” or variations thereof as used herein refers to the ability of a composition to inhibit one or more components of a biological pathway.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

### EXAMPLES

The following examples are illustrative only and are not intended to limit the present invention.

### Example 1—Construction of GLP-1 Vectors

Vectors were constructed in which a leader sequence was placed upstream of one of several GLP-1 receptor agonist amino acid sequences followed by a fusion domain. The resulting protein sequence was back-translated, followed by addition of a kozak consensus sequence, stop codon, and cloning sites. The sequences were produced, and cloned into an expression vector containing a chicken-beta actin promoter with CMV enhancer. The expression construct was flanked by AAV2 ITRs. The feline thrombin-dulaglutide amino acid sequence is shown in SEQ ID NO: 14. The feline thrombin-albiglutide amino acid sequence is shown in SEQ ID NO: 18. The feline GLP-1-SA amino acid sequence is shown in SEQ ID NO: 16.

### Example 2—In Vitro Expression

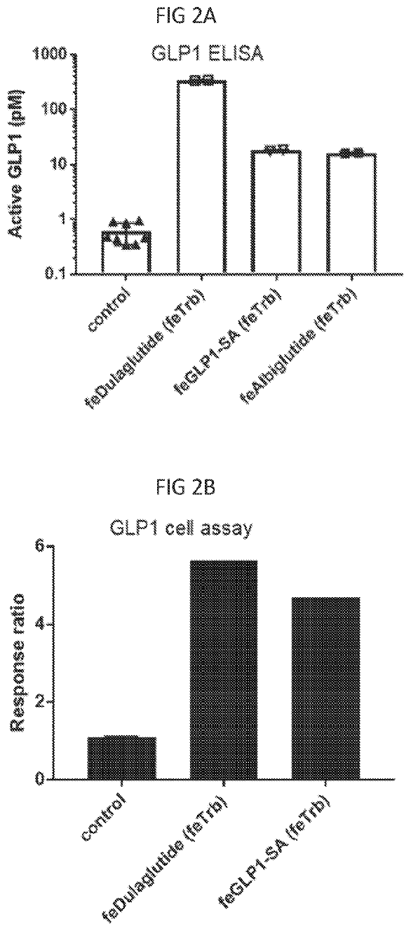

The purified plasmids for the constructs were transfected into triplicate wells of a 6 well plate of 90% confluent HEK 293 cells using lipofectamine 2000 according to the manufacturer's instructions. Supernatant was harvested 48 hours after transfection and active GLP-1 was measured using ELISA specific to active form GLP-1 (7-36). The expression of the three constructs is shown in FIG. 2A. GLP-1 activity in the culture supernatants was measured by cell-based GLP-1 activity assay (GeneBLAzer GLP1R-CRE-bla CHO-KI cell-based assay) (FIG. 2B). The feline dulaglutide construct performed the best in both the expression and activity assays.

### Example 3—Pilot Expression in Rag1 KO Mice

The following constructs were packaged in an AAVrh91 vector by triple transfection and iodixanol gradient purification, as previously described.
AAVrh91.CB7.feDulaglutide(hIL2).rBG, with human IL2 signal
AAVrh91.CB7.feDulaglutide(feIL2).rBG, with feline IL2 signal
AAVrh91.CB7.feDulaglutide(feTrb).rBG
AAVrh91.CB7.feGLP1-SA(feTrb).rBG
AAVrh91.CB7.feAlbiglutide(feTrb).rBG

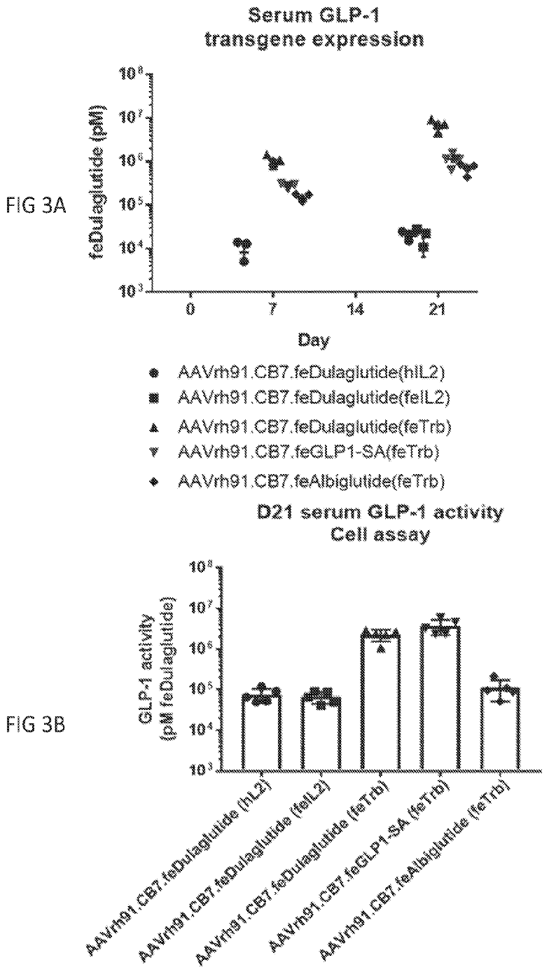

Rag1KO female mice were treated with an intravenous injection of the vector (1×10¹¹ GC/mouse) via IM injection. Serum was serially collected by separating whole blood in serum separator tubes containing 5 microliters DPP-IV inhibitor (Millipore) and assayed for active GLP-1 expression and activity as above. Serum active GLP-1 concentrations are shown in FIG. 3A; activity in FIG. 3B. These data demonstrate that high levels of GLP-1 agonists can be expressed in vivo using AAV delivery technology. In addition, the use of a thrombin prepropeptide leader appears to confer significant advantages to the in vivo expression of feGLP-1-Fc.

### Example 4—Expression and Immunogenicity Study of AAV feGLP-1-Fc and feGLP-1-SA in Cats

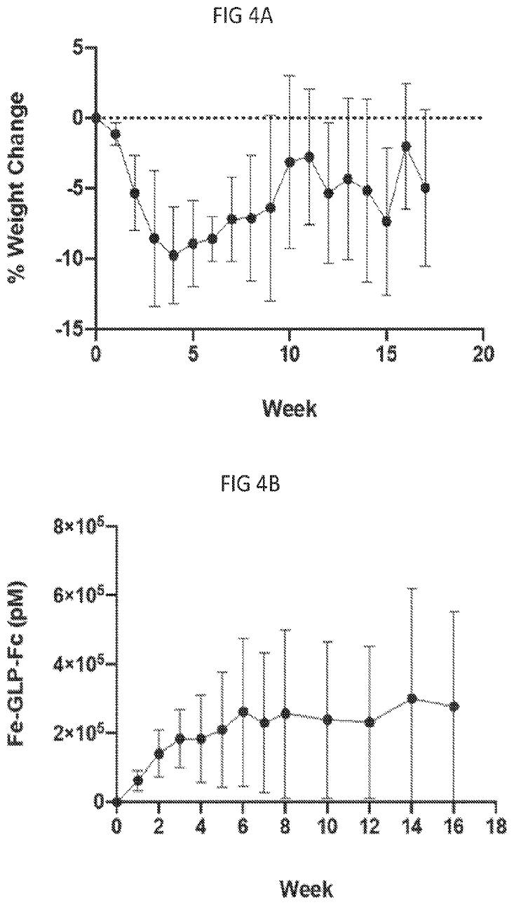

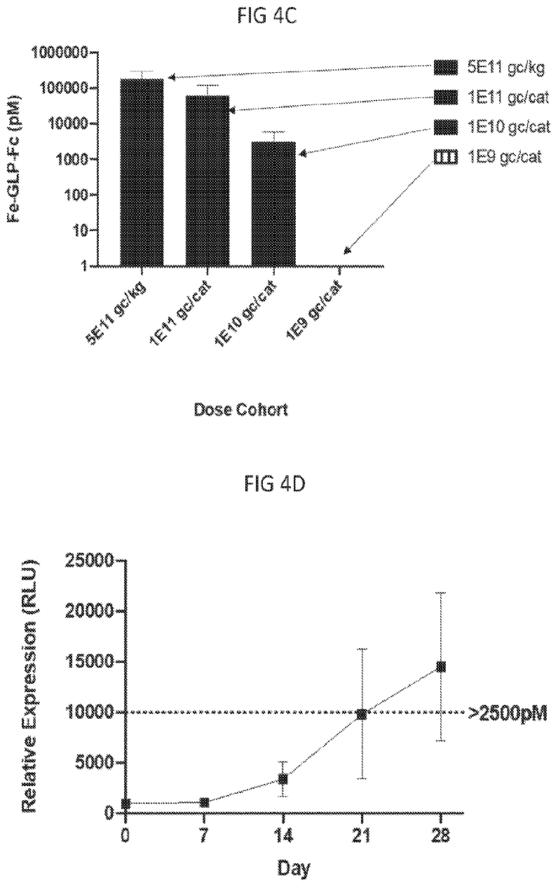

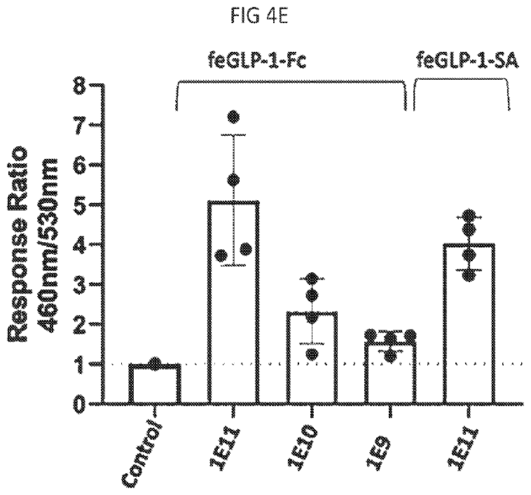

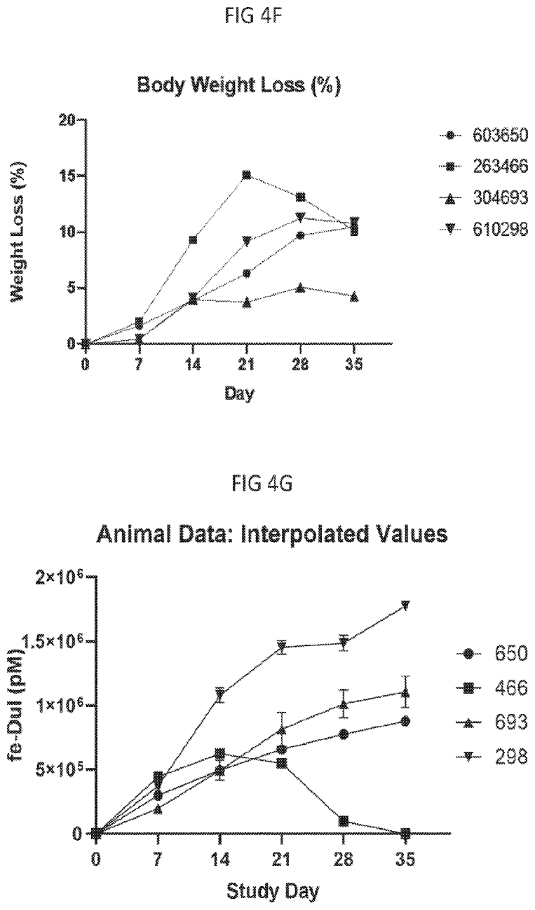

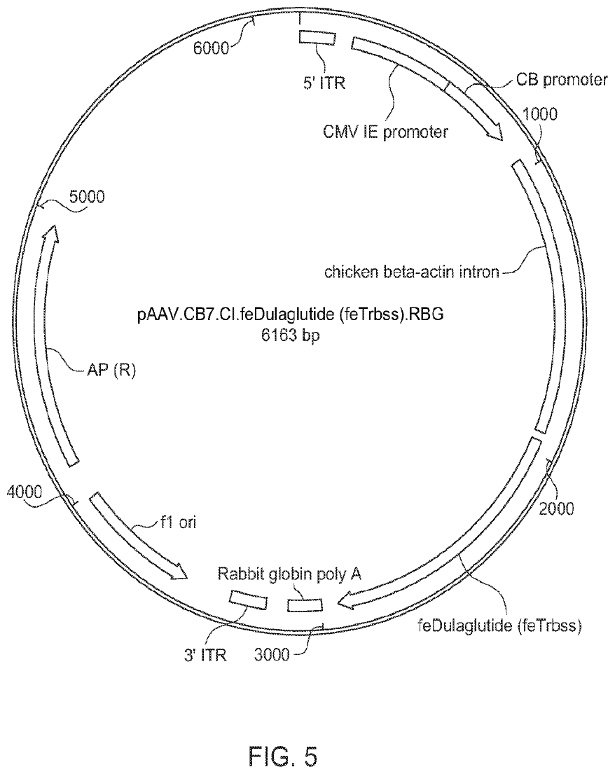

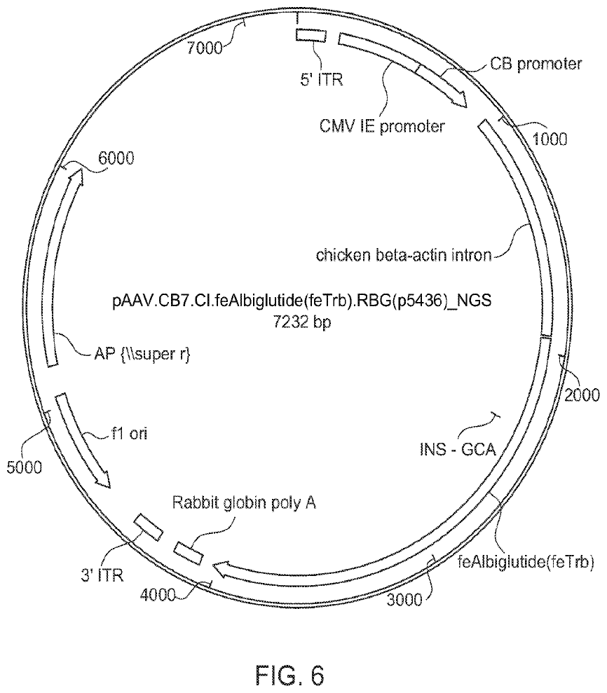

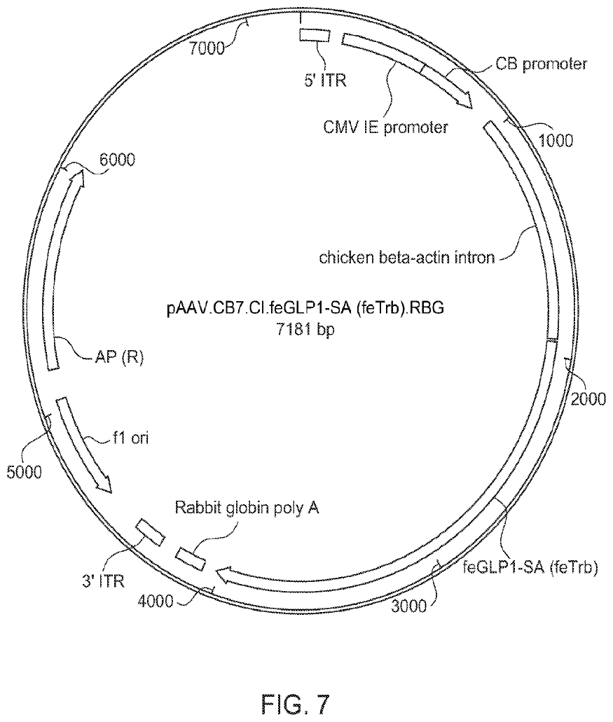

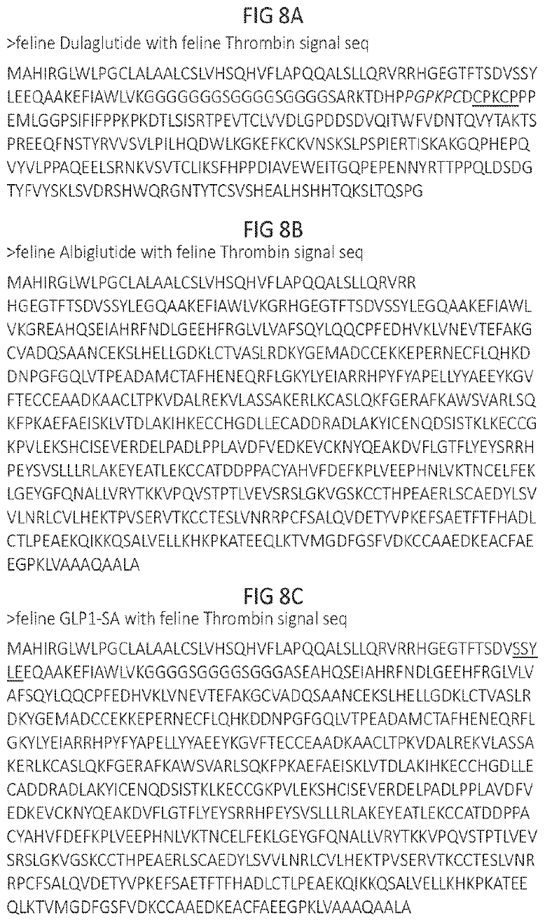

Cats (n=4/cohort) were treated with either 5×10¹¹ GC/kg, 1×10¹¹ GC/cat, 1×10¹⁰ GC/cat or 1×10⁹ GC/cat AAV-feGLP-1-Fc (AAVrh91.CB7.CI.feDulaglutide (feTrbss)), delivered intramuscularly (IM or I.M.). Body weight (FIG. 4A) and GLP-1 expression (FIGS. 4B and C) were recorded. GLP-1 Expression was evaluated using an ELISA specific to the active form of GLP-1 (7-36).

Weight loss was observed (mean˜10%) over the first 4 weeks. Animal started to gain weight over the duration of the study but at week 18 were still, on average 5% lighter than at study start.

Long term expression of feGLP-1-Fc was evaluated over 16 weeks in the high dose (5×10¹¹ GC/kg) group (FIG. 4B). This data shows sustained expression of feGLP-1-Fc that plateaued at around week 6 at a mean concentration of approximately 2.6×10⁵ pM.

By day 28 (D28) mean expression levels for each of the cohorts was 1.8×10⁵ pM (5×10¹¹ GC/kg), 6.5×10⁴ pM (1×10¹¹ GC/cat) and 3.1×10³ pM (1×10¹⁰ GC/cat). Levels of feGLP-1-Fc in the 1×10⁹ GC/cat cohort were below the level of sensitivity for the assay. The mean levels of feGLP-Fc in the top three dose cohorts are above the expected therapeutic dose.

A separate cohort (n=4) of cats was dosed with 1×10¹¹ GC/cat feGLP-1-SA [AAVrh91.CB7.CI.feDulaglutide-SA(feTrb).rBG(p5432)] (FIG. 4D). Good expression of the protein was observed at day 28 where mean serum levels >2.5×10³ pM.

Activity of the GLP-1 agonists in the serum of animals was evaluated using a cell-based GLP-1 activity assay (GeneBLAzer GLP-1R-CRE-bla CHO-KI cell-based assay) (FIG. 4 E). These data demonstrate a clear dose response of GLP-1 activity in the AAV-feGLP-Fc cohorts and that similar activity was seen at the equivalent dose of the AAV-feGLP-1-SA.

### Example 5—Durable Expression and Physiological Benefit after Administration of AAV feGLP-1-SA in Cats with or without Insulin

### Study Design

A study was conducted to evaluate the effectiveness and safety of AAV feGLP-1-SA (recombinant adeno-associated virus vector serotype rh91 containing DNA transgene expressing feline specific GLP-1 serum albumin fusion protein) administered as a single intramuscular (IM) injection, with or without initial insulin therapy, for the management of diabetes mellitus (DM) in cats.

Ten cats were randomly assigned to Arm 1 or Arm 2. At Day 0 cats in both Arms received an intramuscular injection of 1×10¹¹ gene copies/animal of AAV feGLP-1-SA. Cats in Arm 2 also received daily insulin injections starting at Day 0. Cats in Arm 1 were permitted to start insulin if necessary for adequate diabetic control. Investigators were allowed to use their preferred brand of insulin [ProZinc (n=2), Vetsulin (n=2), or Glargine human insulin (n=6)].

Enrolled cats were diabetic but either treatment naïve or previously treated—but not currently receiving insulin or other anti-diabetic drug. Inclusion criteria includes a) at least one clinical sign consistent with DM [polyuria (PU), polydipsia (PD), or unintended weight loss despite a good appetite]; b) fasting blood glucose >270 mg/dL; c] glucosuria; and d) serum fructosamine >400 μmol/L.

Sustained Expression of feGLP-1-SA

Prior to clinical testing, the serum concentration of feGLP-1-SA required for a therapeutic benefit in felines was estimated based on the known value in humans for a recombinant GLP-1-Fc fusion protein, dulaglutide (tradename Trulicity®), which is 800 pM; applying a 20% increase in the target concentration to account for decreased potency of GLP-1-SA in comparative testing of GLP-1-SA and GLP-1-Fc (data not shown). The resulting value, 1000 pM, was multiplied by 3× to account for the possibility that felines might be less sensitive to GLP-1 than humans. Thus, the selected target of 3000 pM represents a conservative estimate for the minimum therapeutically effective concentration of feGLP-1-Fc in serum of a subject feline.

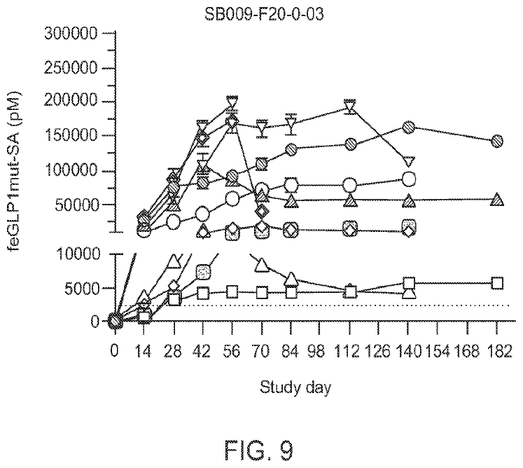

As shown in FIG. 9, all treated cats expressed the feGLP-1-SA protein above the target therapeutic threshold of 3,000 picomolar (pM) throughout the study period of 182 days.

Given that expression levels are roughly constant, continued expression is expected. About half of all cats expressed at feGLPT-SA at a sustained level of about 50,000 pM or greater.

The data demonstrate that all treated cats have expression above the therapeutic threshold concentration for at least 180 days, with 8 cats expressing feGLP-1-SA at least 5-fold above the threshold.

Frustosamine Levels Decreased by AAV feGLP-1-SA with or without Insulin

Fructosamine is a glycated serum protein used by veterinarians to evaluate longer-term diabetic control in cats, similar to use of HbA1c as a marker in humans. As shown in Table 1, cats in both study arms exhibited decreased fructosamine in response to treatment with AAV feGLP-1-SA. Notably, mean fructosamine levels decreased in Arm 1 at day 14 (D14) without administration of insulin. This reduction in fructosamine levels of at least about 9% at Day 14 is due solely to AAV feGLP-1-SA. Sustained decreases in fructosamine levels were observed in both study groups through day 70 (D70).

TABLE 1

| | Mean | | | | | |
|---|---|---|---|---|---|---|
| | Screening | Mean D 14 | Mean D 28 | Mean D 42 | Mean D 56 | Mean D 70 |
| | Fructosamine | Fructosamine | Fructosamine | Fructosamine | Fructosamine | Fructosamine |
| Arm | (μmol/L) | (μmol/L) | (μmol/L) | (μmol/L) | (μmol/L) | (μmol/L) |
| 1 | 508.8 | 463.3 | 481.3 | 425.3 | 333.3 | 364.5 |
| 2 | 446.4 | 423.8 | 401.2 | 419.0 | 397.2 | 367.8 |
| All cats | 474.1 | 441.3 | 436.8 | 421.8 | 368.8 | 366.1 |

To compare efficacy of treatment with AAV feGLP-1-SA and insulin together to treatment with insulin alone, results were compared to publicly available data on two branded insulin products, ProZinc® and Vetsulin® (available at animaldrugsatfda.fda.gov). Data for study subjects are reported as days from commencement of insulin administration (day 0 in Arm 2; various days in Arm 1). As shown in Table 2, after 14 days, subjects previously injected with AAV feGLP-1-SA have lower fructosamine than cats treated with insulin alone (based on historic data for the two veterinary approved insulins, ProZinc® and Vetsulin®). In sum, the fructosamine data demonstrates that AAV feGLP-1-SA not only is effective alone but also improves overall diabetic control with insulin compared to insulin monotherapy.

TABLE 2

| | | | | AAV feGLP-1-SA plus |
|---|---|---|---|---|
| | PROZINC ® | | VETSULIN ® | Insulin |
| | (no. of subjects) | | (no. of subjects) | (no. of subjects) |
| DAY | historic control | | historic control | study data |
| 0 | 505.9 | (151) | 607.3 (77) | 473 (9) |
| 7 | n/a | | n/a | |
| 14 | 456.1 | (150) | n/a | |
| 30 | 408.4 | (150) | 487.8 (75) | 386 (8)** |
| 45 | 380.7 | (143) | n/a | |

| | | |
|---|---|---|
| 60 | n/a | 453.7 (75) |
| 180 | n/a | 454.8 (72) |

Blood Glucose Levels Decreased by AAV feGLP-1-SA with or without Insulin

Blood glucose levels are a direct measurement of diabetes control and was measured at each visit. At day 42 and day 84 visits, insulin was withheld 12 hours prior to the blood glucose measurements prior to the first measurement of a complete 9-hour blood glucose curve. All other days were single measurements within 1 hour following the morning insulin, if the cat was on insulin. Table 3 shows mean blood glucose change from DO for each animal. AAV feGLP-1-SA without insulin (Arm 1, D14 and D28) or with insulin (other values) causes decreases in glucose levels.

TABLE 3

| Change in mean | | | | | | | |
|---|---|---|---|---|---|---|---|
| Glucose at Visit: | D 14 | D 28 | D 56 | D 70 | D 112 | D 140 | D 182 |
| All Arm 1 | −90.3 | −97.5 | −148.5 | −278.3 | −148.0 | −107.3 | −218.3 |
| All Arm 2 | −92.8 | −129.2 | −122.8 | −153.1 | −282.6 | −320.4 | −323.6 |
| All Cats | −91.7 | −115.1 | −134.2 | −216.1 | −215.7 | −214.2 | −271.3 |

Insulin Dose Requirements Decreased by AAV feGLP-1-SA

Remission is defined as the ability to cease insulin treatment when the cat no longer exhibits clinical signs of diabetes and has normal blood glucose levels. One of the subjects in Arm 1 entered remission at day 70 and remained in remission through the end of the study. Another subject was able to be removed from insulin from D54 to D84 and therefore was in remission for one month. Two subjects in Arm 2 completed the study on very low doses of insulin, 1 IU twice daily, suggesting they may be close to remission but at the least are able to be controlled with a dose far lower than typical. The average Insulin Dose at actual study Days 30, 42 and 60 are listed in Table 4 below and compared to the Vetsulin® and ProZinc® historic data at the same time frame in Table 5.

The comparison between study data and historic data in Table 5 shows that treatment AAV feGLP-1-SA permits insulin dosages to be reduced below average doses for cats receiving insulin alone without AAV feGLP-1-SA.

TABLE 4

| | Average of | Average of | Average of | Average of |
|---|---|---|---|---|
| | Dose on | Dose on | Dose on | Dose at |
| Subject | D 30 | D 42 | D 60 | D 182 |
| 1 | 4 | 5 | 3 | 1 |
| 2 | 1 | 1.5 | 1.5 | 2 |
| 3 | 1 | 2 | 0 | 2 |
| 4 | 2 | 3 | 3 | 1 |
| 5 | 2 | 1 | 1 | 0 |
| 6 | 1 | 1 | 1 | 5.5 |
| 7 | 3 | 3 | 4 | N/A |
| 8 | 3 | 4 | 5 | 5 |
| 9 | 5 | 5 | 5 | 5 |
| Mean | 2.4 | 2.8 | 2.6 | 2.7 |

TABLE 5

| | Mean Insulin | Mean Insulin | Mean Insulin |
|---|---|---|---|
| Product | Dose Day 30 | Dose Day 42 | Dose Day 60 |
| AAV feGLP-1-SA | 2.4 | 2.8 | 2.6 |
| and Insulin | | | |
| (study data) | | | |
| Vetsulin ® | 3.2 | — | 3.6 |
| (historic data) | | | |
| Prozinc ® | 3.6 | 3.7 | |
| (historic data) | | | |

### Example 7—Durable Expression after Administration of AAV feGLP-1-Fc in Cats

A study was conducted to evaluate the effectiveness and safety of AAV feGLP-1-Fc (recombinant adeno-associated virus vector serotype rh9l containing DNA transgene expressing feline specific GLP-1 Fc receptor domain fusion protein) administered as a single intramuscular (IM) injection.

Four cats each received an intramuscular injection of 5×10¹¹ gene copies/animal of AAV feGLP-1-Fc. Expression of the transgene was measured in blood plasma every 14 days.

Prior to clinical testing, the inventors estimated the serum concentration of feGLP-1-Fc required for a therapeutic benefit in felines based on the known value in humans for a recombinant GLP-1-Fc fusion protein, dulaglutide (tradename Trulicity®), which is 800 pM. This value, 800 pM, was multiplied by 3× to account for the possibility that felines might be less sensitive to GLP-1 than humans. Thus, the inventors' selected target of 2400 pM represents a conservative estimate for the minimum therapeutically effective concentration of feGLP-1-Fc in serum of a subject feline.

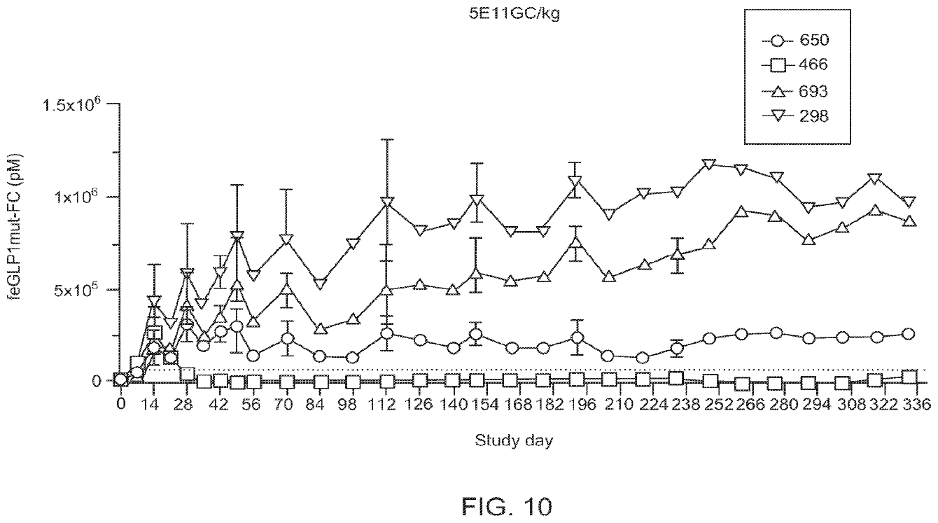

As shown in FIG. 10, three of four animals expressed high levels of feGLP-1-Fc, well above the 2,400 pM therapeutic efficacy threshold, for over 330 days.

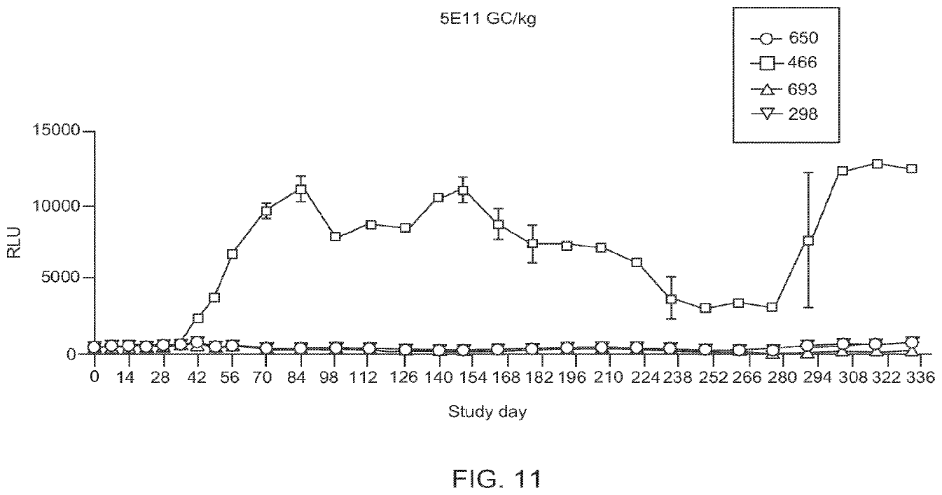

One animal developed antibodies against the transgenic protein as shown in FIG. 11, consistent with the low levels of expressed protein observed in that animal.

These data demonstrate that a single injection of AAV feGLP-1-Fc causes durable expression of the transgenic protein at high levels.

### Example 8—Durable Expression after Administration of AAV feGLP-1-SA in Healthy Cats

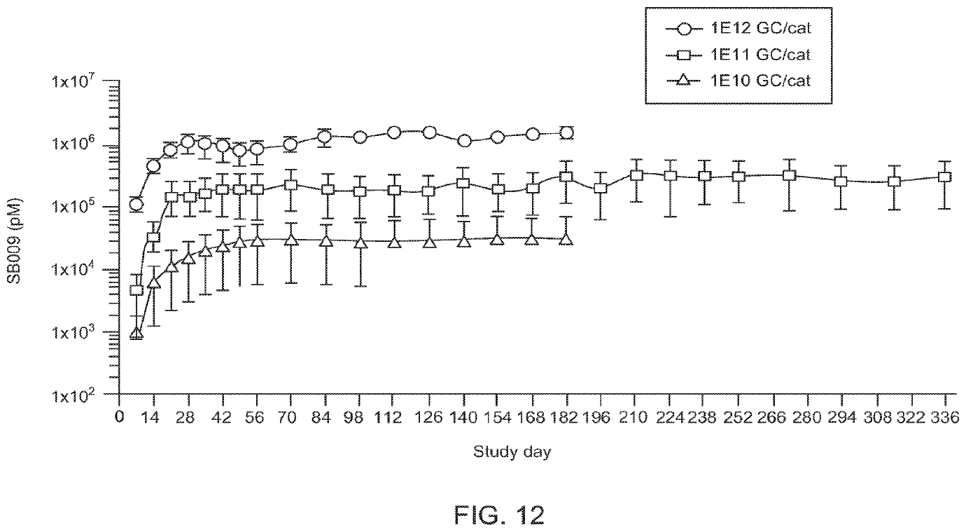

A study was conducted to evaluate the effectiveness and safety of AAV feGLP-1-SA (recombinant adeno-associated virus vector serotype rh91 containing DNA transgene expressing feline specific GLP-1 serum albumin fusion protein) administered as a single intramuscular (IM) injection. 16 cats each received an intramuscular injection of AAV feGLP-1-SA at one of three dose levels: 1e10, 1e11, or 1e12 gene copies/animal. Expression of the transgene was measured in blood plasma every 14 days. As shown in FIG. 12, all sixteen animals expressed high levels of GLP-1-Fc, well above the 3,000 pM therapeutic efficacy threshold, for over 330 days. No animals developed antibody to the transgene protein.

These data demonstrate that a single injection of AAV feGLP-1-SA causes durable expression of the transgenic protein at high levels.

### Specific Embodiments

1. A viral vector comprising a nucleic acid comprising a polynucleotide sequence encoding a fusion protein comprising (a) a leader sequence comprising a secretion signal peptide, (b) a glucagon-like peptide-1 (GLP-1) receptor agonist, and (c) a fusion domain comprising either (i) a feline IgG Fc or a functional variant thereof or (ii) a feline albumin or a functional variant thereof.
2. The viral vector according to embodiment 1, wherein the vector is an adeno-associated viral vector.
3. The viral vector according to embodiment 1 or embodiment 2, wherein (i) the secretion signal peptide of the leader sequence comprises a feline thrombin signal peptide; (ii) the leader sequence comprises a feline thrombin propeptide; and/or (iii) the leader sequence comprises a feline thrombin leader sequence.
4. The viral vector according to embodiment 3, wherein (i) the signal peptide of the leader sequence comprises the polypeptide sequence MAHIRGLWLPGCLALAALCSLVHS (SEQ ID NO: 8) or a functional variant thereof having at most 1, 2, or 3 amino acid substitutions; (ii) the leader sequence comprises the polypeptide sequence QHVFLAPQQALSLLQRVRR (SEQ ID NO: 9) or a functional variant thereof having at most 1, 2, or 3 amino acid substitutions; and/or (iii) the leader sequence comprises the polypeptide sequence MAHIRGLWLPGCLALAALCSLVHSQHVFLAPQQALSLLQRVRR (SEQ ID NO: 7) or a functional variant thereof having at most 1, 2, or 3 amino acid substitutions.
5. The viral vector according to embodiment 1 or embodiment 2, wherein the leader sequence comprises a feline IL-2 leader sequence.
6. The viral vector according to embodiment 5, wherein the feline IL-2 leader sequence comprises a sequence the sequence of MYKIQLLSCIALTLILVTNS (SEQ ID NO: 10) or a functional variant thereof having at most 1, 2, or 3 amino acid substitutions.
7. The viral vector according to any one of embodiments 1 to 6, wherein the GLP-1 receptor agonist is feline GLP-1(7-37) or a functional variant thereof.
8. The viral vector according to embodiment 7, wherein the GLP-1 receptor agonist is a DPP-IV resistant variant of feline GLP-1(7-37).
9. The viral vector according to embodiment 7 or 8, wherein the GLP-1 receptor agonist is HGEGTFTSDVSSYLEEQAAKEFIAWLVKGGG (SEQ ID NO: 3) or a functional variant thereof having at most 1, 2, or 3 amino acid substitutions.
10. The viral vector of embodiment 9, wherein the GLP-1 receptor agonist comprises a glycine (G) at position 8 relative to feline GLP-1(1-37) and/or a glutamine (E) at position 22 relative to GLP-1(1-37), wherein optionally the GLP-1 receptor agonist is HGEGTFTSDVSSYLEEQAAKEFIAWLVKGGG (SEQ ID NO: 3).
11. The viral vector according to embodiment 7 or 8, wherein the GLP-1 receptor agonist is HAEGTFTSDVSSYLEGQAAKEFIAWLVKGRG (SEQ ID NO: 2), HGEGTFTSDVSSYLEGQAAKEFIAWLVKGRG (SEQ ID NO: 4), or a functional variant thereof having at most 1, 2, or 3 amino acid substitutions.
12. The viral vector according to any one of embodiments 1 to 6, wherein the GLP-1 receptor agonist is GLP-1(1-37) or a functional variant thereof.
13. The viral vector according to any one of embodiments 1 to 6, wherein the GLP-1 receptor agonist is HGEGTFTSDVSSYLEGQAAKEFIAWLVKGRG (SEQ ID NO: 4), or a functional variant thereof, optionally sharing at least 90% or 100% identity to SEQ ID NO: 4.
14. The viral vector according to any one of embodiments 1 to 13, wherein the fusion protein comprises a second GLP-1 receptor agonist.
15. The viral vector according to embodiment 14, wherein the GLP-1 receptor agonist is two tandem copies of GLP-1(7-37) or a DPP-IV resistant variant thereof.
16. The viral vector according to embodiment 15, wherein the DPP-IV resistant variant is HGEGTFTSDVSSYLEGQAAKEFIAWLVKGRG (SEQ ID NO:4).
17. The viral vector according to any one of embodiments 1 to 16, wherein the fusion protein further comprises a linker between the GLP-1 receptor agonist and the fusion domain.
18. The viral vector according to any one of embodiments 1 to 17, wherein the fusion domain is a feline IgG Fc or a functional variant thereof.
19. The viral vector according to embodiment 18, wherein the fusion domain is a feline IgG Fc.
20. The viral vector according to embodiment 18 or 19, wherein the feline IgG Fc is a feline IgG2 Fc.
21. The viral vector according to embodiment 18 or 19, wherein the feline IgG Fc is a feline IgG1a Fc.
22. The viral vector according to embodiment 18 or 19, wherein the feline IgG Fc a feline IgG1b Fc.
23. The viral vector of embodiment 18 or embodiment 19, wherein the feline IgG Fc shares at least 90% identity, at least 95% identity, at least 99% identity, or at least 100% identity to SEQ ID NO: 11.
24. The viral vector according to any one of embodiments 1 to 17, wherein the fusion domain is a feline albumin or a functional variant thereof.
25. The viral vector according to embodiment 18, wherein the fusion domain is a feline albumin.
26. The viral vector of embodiment 24 or embodiment 25, wherein the feline albumin shares at least 90% identity, at least 95% identity, at least 99% identity, or at least 100% identity to SEQ ID NO: 12.
27. The viral vector of embodiment 1, wherein the fusion protein comprises (a) feline thrombin leader, (b) a DPP-IV resistant variant of GLP-1(7-37), a linker, and (c) a feline IgG Fc.
28. The viral vector according to embodiment 1 or embodiment 26, wherein the fusion protein has the sequence of SEQ ID NO: 14, or a sequence at least 90%, at least 95%, or at least 98% identical thereto.
29. The viral vector according to embodiment 1 or 26 to 27, wherein the sequence encoding the fusion protein is SEQ ID NO: 15 or a sequence at least 90%, at least 95%, or at least 98% identical thereto.
30. The viral vector of embodiment 1, wherein the fusion protein comprises (a) feline thrombin leader, (b) a DPP-IV resistant variant of GLP-1(7-37), a linker, and (c) a feline albumin.
31. The viral vector according to embodiment 1 or embodiment 30, wherein the fusion protein has the sequence of SEQ ID NO: 16, or a sequence at least 90%, at least 95%, or at least 98% identical thereto.
32. The viral vector according to any one of embodiment 1 or 30 to 31, wherein the sequence encoding the fusion protein is SEQ ID NO: 17 or a sequence at least 90%, at least 95%, or at least 98% identical thereto.
33. The viral vector of embodiment 1, wherein the fusion protein comprises (a) feline thrombin leader, (b) two tandem copies of feline GLP-1(7-37) or a DPP-IV resistant variant thereof, a linker, and (c) a feline albumin.
34. The viral vector according to any one of embodiments 1 or 33, wherein the fusion protein has the sequence of SEQ ID NO: 18 or SEQ ID NO: 20, or a sequence at least 90%, at least 95%, or at least 98% identical thereto.
35. The viral vector according to any one of embodiments 1 or 33 to 34, wherein the sequence encoding the fusion protein is SEQ ID NO: 19 or a sequence at least 90%, at least 95%, or at least 98% identical thereto.
36. The viral vector according to any of embodiments 1 to 35, comprising:
   (a) an AAV capsid, and
   (b) a vector genome packaged in the AAV capsid, said vector genome comprising AAV inverted terminal repeats (ITRs), the polynucleotide sequence encoding the fusion protein, and regulatory sequences which direct expression of the fusion protein.
37. The viral vector according to any of embodiments 1 to 35, comprising:
   (a) an AAV capsid, and
   (b) a vector genome packaged in the AAV capsid, said vector genome comprising AAV inverted terminal repeats (ITRs), the polynucleotide sequence encoding the fusion protein, and regulatory sequences which direct insertion of the polynucleotide sequence encoding the fusion protein to the genome of a host cell.
38. The viral vector according to any one of embodiments 1 to 37, wherein the viral vector is a recombinant adeno-associated virus (rAAV) having the capsid of AAV8 or a functional variant thereof.
39. The viral vector according to any one of embodiments 1 to 37, wherein the viral vector is an rAAV having the capsid of AAVrh91 or a functional variant thereof.
40. The viral vector according to any one of embodiments 1 to 37, wherein the viral vector is an rAAV having the capsid of AAV3B.AR2.12 or a functional variant thereof.
41. The viral vector according to any one of embodiments 1 to 37, wherein the viral vector is an rAAV having a capsid selected from AAV9, AAVrh64R1, AAVhu37, or AAVrh10.
42. A pharmaceutical composition suitable for use in treating a metabolic disease in a feline comprising an aqueous liquid and the viral vector according to any of embodiments 1 to 41.
43. The viral vector according to any one of embodiments 1 to 41 or the pharmaceutical composition according to embodiment 42 for use in a method for treating a feline subject having a metabolic disease, optionally diabetes.
44. Use of the viral vector according to any one of embodiments 1 to 41 or the pharmaceutical composition according to embodiment 42 in the manufacture of a medicament for treating a feline subject having a metabolic disease, optionally diabetes.
45. The viral vector or use according to embodiment 43 or 44, wherein the composition is formulated to be administered to the feline subject at a dose of 1×10⁹ GC/kg to 3×10¹³ GC/kg of the rAAV and/or wherein the rAAV is delivered intramuscularly or intravenously.
46. A method of treating a feline subject having a metabolic disease, comprising administering to the feline subject an effective amount of the viral vector according to any one of embodiments 1 to 41 or the pharmaceutical composition according to embodiment 42.
47. The method according to embodiment 46, wherein the metabolic disease is diabetes.
48. The method according to embodiment 47, wherein the diabetes is Type 1 diabetes.
49. The method according to embodiment 47, wherein the diabetes is Type 2 diabetes.
50. The method according to any one of embodiments 45 to 49, wherein the effective amount is administered intravenously.
51. The method according to any one of embodiments 45 to 49, wherein the effective amount is administered intramuscularly.
52. The method according to any one of embodiments 45 to 51, wherein the effective amount is between 1×10⁹ GC/kg to 3×10¹³ GC/kg body mass of the rAAV
53. The method according to any one of embodiments 45 to 51, wherein the effective amount is between 1×10¹⁰ GC/kg to 3×10¹³ GC/kg body mass of the rAAV.
54. The method according to any one of embodiments 54 to 51, wherein the method results in expression of the fusion protein in the serum of the subject for at least three months, at least six months, or at least twelve months.
55. The method according to any one of embodiments 54 to 51, wherein the method results in expression of the fusion protein in the subject at a serum concentration of at least 3,000 picomolar (pM), at least 5,000 pM, at least 10,000 pm, at least 25,000 pM, or at least 50,000 pM for at least three months, at least six months, or at least twelve months.
56. The method according to any one of embodiments 54 to 51, wherein the method results in expression of the fusion protein in the subject at a serum concentration of 3,000 picomolar (pM) to 200,000 pM, 5,000 picomolar (pM) to 200,000 pM, 10,000 picomolar (pM) to 200,000 pM, 25,000 picomolar (pM) to 200,000 pM, or 50,000 picomolar (pM) to 200,000 pM, for 3-12 months, 6-12 months, or twelve months.
57. The method according to any one of embodiments 54 to 51, wherein the method results in expression of the fusion protein in the subject at a therapeutically effective concentration for at least three months, at least six months, or at least twelve months.
58. The method according to any one of embodiments 54 to 57, wherein the method decreases serum fructosamine in the subject by about 6%.
59. The method according to any one of embodiments 54 to 57, wherein the method decreases serum fructosamine in the subject by 5%-10%.
60. The method according to any one of embodiments 54 to 59, wherein the method decreases serum glucose in the subject by about 10%.
61. The method according to any one of embodiments 54 to 60, wherein the method decreases serum glucose in the subject by 10%-20%.
62. The method according to any one of embodiment 54 to 61, wherein the method comprises administering an effective amount of insulin to the subject.
63. The method according to embodiment 61, wherein the insulin is protamine zinc recombinant human insulin (ProZinc®), porcine insulin zinc suspension (Vetsulin®), and/or insulin glargine (Lantus®).
64. The method according to claim 61 of claim 63, wherein the method decreases insulin dose requirements compared to insulin dose requirements for the subject before treatment with the viral vector.
65. The method according to any one of claims 54 to 64, wherein the method causes remission of the metabolic disease in the subject.

### Sequence Listing Free Test

The following information is provided for sequences containing free text under numeric identifier <223>.

| SEQ ID NO: | |
|---|---|
| (containing free text) | Free text under <223> |
| 3 | <223> Constructed sequence |
| 4 | <223> Constructed sequence |
| 5 | <223> Constructed sequence |
| 6 | <223> Constructed sequence |
| 13 | <223> Constructed sequence |
| 14 | <223> Constructed sequence |
| 15 | <223> Constructed sequence |
| 16 | <223> Constructed sequence |
| 17 | <223> Constructed sequence |
| 18 | <223> Constructed sequence |
| 19 | <223> Constructed sequence |
| 20 | <223> Constructed sequence |
| 21 | <223> Constructed sequence |
| 22 | <223> Constructed sequence |
| 23 | <223> Constructed sequence |
| 25 | <220> |
| | <221> misc_feature |
| | <222> (1) . . . (2211) |
| | <223> AAVrh91 |

All documents cited in this specification, are incorporated herein by reference. U.S. Provisional Patent Application No. 63/069,492, filed Aug. 24, 2020 is incorporated herein by reference in its entireties, together with its sequence listing. The sequence listing filed herewith labeled “20-9292PCT_Seq-Listing_ST25” and the sequences and the text therein are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated viral (rAAV) vector comprising a nucleic acid comprising a polynucleotide sequence encoding a fusion protein comprising (a) a leader sequence comprising a secretion signal peptide, (b) a glucagon-like peptide-1 (GLP-1) receptor agonist, and (c) a fusion domain comprising either (i) a feline IgG Fc or a functional variant thereof or (ii) a feline albumin or a functional variant thereof.

2. (canceled)

3. The rAAV vector according to claim 1, wherein (i) the secretion signal peptide of the leader sequence comprises a feline thrombin signal peptide; (ii) the leader sequence comprises a feline thrombin propeptide; and/or (iii) the leader sequence comprises a feline thrombin leader sequence.

4. The rAAV vector according to claim 3, wherein (i) the signal peptide of the leader sequence comprises the polypeptide sequence SEQ ID NO: 8 or a functional variant thereof having at most 1, 2, or 3 amino acid substitutions; (ii) the leader sequence comprises the polypeptide sequence SEQ ID NO: 9or a functional variant thereof having at most 1, 2, or 3 amino acid substitutions; and/or (iii) the leader sequence comprises the polypeptide sequence SEQ ID NO: 7 or a functional variant thereof having at most 1, 2, or 3 amino acid substitutions.

5. The rAAV vector according to claim 1, wherein the leader sequence comprises a feline IL-2 leader sequence comprising the sequence of SEQ ID NO: 10 or a functional variant thereof having at most 1, 2, or 3amino acid substitutions.

6. (canceled).

7. The rAAV vector according to claim 1, wherein the GLP-1 receptor agonist is feline GLP-1(7-37) or a functional variant thereof.

8. (canceled)

9. The rAAV vector according to claim 7, wherein the GLP-1 receptor agonist is SEQ ID NO: 3 or a functional variant thereof having at most 1, 2, or 3 amino acid substitutions.

10. (canceled)

11. The rAAV vector according to claim 7, wherein the GLP-1 receptor agonist is SEQ ID NO: 2, SEQ ID NO: 4, or a functional variant of SEQ ID NO: 2 or SEQ ID NO: 4 thereof having at most 1, 2, or 3 amino acid substitutions.

12-13 (canceled)

14. The rAAV vector according to claim 1, wherein the fusion protein comprises a second GLP-1 receptor agonist.

15-16. (canceled)

17. The rAAV vector according to claim 1, wherein the fusion protein further comprises a linker between the GLP-1 receptor agonist and the fusion domain, wherein the fusion domain is a feline IgG Fc.

18-19. (canceled)

20. The viral vector according to claim 17, wherein the feline IgG Fc is an
(i) IgG2 Fc;
(i) IgG1a Fc; or
(i) IgG2b Fc.

21-22. (canceled)

23. The viral vector according to claim 17, wherein the feline IgG Fc shares at least 90% identity, at least 95% identity, at least 99% identity, or at least 100% identity to SEQ ID NO: 11.

24-26. (canceled)

27. The rAAV vector of claim 1, wherein the fusion protein comprises (a) feline thrombin leader, (b) a DPP-IV resistant variant of GLP-1(7-37), a linker, and (c) a feline IgG Fc.

28. The rAAV vector according to claim 1, wherein the fusion protein has the sequence of SEQ ID NO: 14, or a sequence at least 90%, at least 95%, or at least 98% identical thereto.

29 (canceled)

30. The rAAV vector of claim 1, wherein the fusion protein comprises (a) feline thrombin leader, (b) a DPP-IV resistant variant of GLP-1(7-37), a linker, and (c) a feline albumin.

31. The rAAV vector according to claim 1, wherein the fusion protein has the sequence of SEQ ID NO: 16, or a sequence at least 90%, at least 95%, or at least 98% identical thereto.

32-33 (canceled)

34. The rAAV vector according to claim 1, wherein the fusion protein has the sequence of SEQ ID NO: 18 or SEQ ID NO: 20, or a sequence at least 90%, at least 95%, or at least 98% identical thereto.

35. (canceled)

36. The rAAV vector according to claim 1, comprising:
(a) an AAV capsid, and
(b) a vector genome packaged in the AAV capsid, said vector genome comprising AAV inverted terminal repeats (ITRs), the polynucleotide sequence encoding the fusion protein, and regulatory sequences which direct expression of the fusion protein or regulatory sequences which direct insertion of the polynucleotide sequence encoding the fusion protein to the genome of a host cell.

37. (canceled)

38. The rAAV vector according to claim 36, wherein the viral vector is a recombinant adeno-associated virus (rAAV) having a capsid selected from AAV8, AAVrh91, AAV3B. AR2.12, AAV9, AAVrh64R1, AAVhu37, or AAVrh10, or a functional variants thereof.

39-41. (canceled)

42. A pharmaceutical composition suitable for use in treating a metabolic disease in a feline comprising an aqueous liquid and the rAAV vector according to claim 1.

43-45. (canceled)

46. A method of treating a feline subject having a metabolic disease, comprising administering to the feline subject an effective amount of the viral vector according to claim 1, wherein the effective amount is between 1×10⁹ GC/kg to 3×10¹³ GC/kg body mass of the rAAV or between 1×10¹⁰ GC/kg to 3×10¹³ GC/kg body mass of the rAAV and further wherein the effective amount is administered intravenously or intramuscularly.

47-53. (canceled)
